(19) United States
(12) Patent Application Publication
(10) Pub. No.: US 2014/0056918 A1
(43) Pub. Date: Feb. 27, 2014

(54) OSCAR ANTAGONISTS

(71) Applicant: NOVO NORDISK A/S, BAGSVAERD (DK)

(72) Inventors: Li Guo, Beijing (CN); Svetlana Panina, Koebenhavn (DK); Jesper Pass, Alleroed (DK); Louise Hjerrild Zeuthen, Birkeroed (DK); Louise Maymann Nitze, Frederiksberg (DK)

(21) Appl. No.: 14/067,526

(22) Filed: Oct. 30, 2013

Related U.S. Application Data
(63) Continuation of application No. PCT/EP2012/064105, filed on Jul. 18, 2012.
(60) Provisional application No. 61/662,599, filed on Jun. 21, 2012.
(60) Provisional application No. 61/524,427, filed on Aug. 17, 2011.

(30) Foreign Application Priority Data
Jul. 18, 2011 (CN) PCT/CN2011/001172
May 28, 2012 (CN) PCT/CN2012/076139

(51) Int. Cl. C07K 16/18

(52) U.S. Cl. CPC C07K 16/18
USPC 424/172.1; 530/389.1

(57) ABSTRACT

The present invention relates to antagonistic OSCAR ligands. The present invention furthermore relates to use of such ligands.

### CROSS-REFERENCES TO RELATED APPLICATIONS

The present application is a continuation of PCT/EP2012/064105, filed Jul. 18, 2012 which claims priority under 35 U.S.C. §119(e) to U.S. Provisional Patent Application No. 61/524,427, filed Aug. 17, 2011 and U.S. Provisional Patent Application No. 61/662,599, filed Jun. 21, 2012, each of which are hereby incorporated by reference in their entirety for all purposes. The present application also claims priority of PCT/CN2011/001172, filed Jul. 18, 2011 and PCT/CN2012/076139, filed May 28, 2012, each of which are hereby incorporated by reference in their entirety for all purposes.

### TECHNICAL FIELD

The present invention relates to compounds useful in treatment and prophylaxis of diseases such as autoimmune inflammatory diseases and bone resorption related disorders. The present invention furthermore relates to OSCAR antagonists as well as use of such compounds.

### BACKGROUND

Osteoclast-associated receptor (OSCAR) is an activating receptor expressed on osteoclasts and myeloid-derived cells, including monocytes, macrophages, neutrophils and dendritic cells. It was reported that OSCAR is up-regulated in the synovial tissue as well as in circulating CD14⁺ cells from rheumatoid arthritis patients. Triggering of OSCAR signalling leads to secretion of pro-inflammatory cytokines and chemokines from monocytes and dendritic cells, promotes differentiation of osteoclasts and hence may be causally involved in the pathogenesis of the inflammation-induced bone resorption. Collagens type I, II, Ill, and IV have been shown to act as ligands for OSCAR (WO2010040998).

No OSCAR antagonists suitable for therapeutic use in patients with diseases associated with bone resorption, e.g. rheumatoid arthritis or other inflammatory diseases have been disclosed thus far.

### SUMMARY

The present invention thus relates to OSCAR antagonists, preferably OSCAR antibodies, as well as use of such compounds in treatment of various diseases such as e.g. autoimmune inflammatory diseases. Such antagonists have the ability to reduce inflammation and/or the ability to modulate osteoclast-mediated bone resorption.

The present invention furthermore relates to methods for inducing collagen dependent maturation of monocyte-derived Dendritic Cells.

### DESCRIPTION

The present invention relates to OSCAR antagonists that are useful in connection with treatment of patients suffering from autoimmune inflammatory diseases such as e.g. rheumatoid arthritis. The compounds of the invention may also be used in treatment of other disorders associated with bone resorption.

### BRIEF DESCRIPTION OF DRAWINGS

FIG. 1: Anti-OSCAR mAb 13F5A5 inhibit osteoclast formation from normal monocytes

### DEFINITIONS

OSCAR: the osteoclast-associated receptor (OSCAR) is a member of the leukocyte receptor complex (LRC) protein family that plays critical roles in the regulation of both innate and adaptive immune responses. OSCAR is expressed in pre-osteoclasts and mature osteoclasts (OC) and promotes OC differentiation from the bone marrow derived precursors. In contrast to mouse OSCAR which expression is restricted to OC, the human receptor is also found in monocytes, macrophages, dendritic cells and neutrophils. In the latter cell types triggering of OSCAR can lead to secretion of pro-inflammatory mediators and promote differentiation of OC from monocyte-derived precursors. The best studied isoform of the OSCAR protein (further referred as “OSCAR”, encoded by mRNA with Genebank accession number AF391162), is a single-span trans-membrane protein comprising 263 amino acids residues (SEQ ID NO 1). It associates with FcRγ which serves as a signalling subunit of the receptor complex. Sequences of six additional putative OSCAR isoforms can be found in various databases (Uniprot accession number Q81YS5-1, Q81YS5-2, Q81YS5-3, Q81YS5-4, Q81YS5-5, Q81YS5-6, Q81YS5-7). All isoforms seem to be resulting from the alternative splicing of a common RNA precursor. Some isoforms are predicted to lack a transmembrane domain due to a frame shift mutation and thus encode for putative secreted proteins. Recombinant soluble proteins comprising e.g. the extracellular domain of OSCAR(OSCAR-ECD) and naturally occurring isoforms OSCAR-S1a and OSCAR-S1b can be generated. The amino acid sequence of human OSCAR-ECD is shown in SEQ ID NO 2. The amino acid sequence of human OSCAR-S1a is shown in SEQ ID NO 3. The amino acid sequence of human OSCAR-S1b is shown in SEQ ID NO 4. “OSCAR variants” refer to proteins having OSCAR activity but comprise one or more alterations in comparison with wt OSCAR, such as e.g. one or more amino acid substitutions/deletions/additions.

SEQ ID NO 1: Human OSCAR (putative signal peptide marked with bold)
MALVLILQLLTLWPLCHTDITPSVPPASYHPKPWLGAQPATVVTPGVNVTLRCRA
PQPAWRFGLFKPGEIAPLLFRDVSSELAEFFLEEVTPAQGGSYRCCYRRPDWGPGVWSQP
SDVLELLVTEELPRPSLVALPGPVVGPGANVSLRCAGRLRNMSFVLYREGVAAPLQYRHSA
QPWADFTLLGARAPGTYSCYYHTPSAPYVLSQRSEVLVISWEDSGSSDYTRGNLVRLGLAG
LVLISLGALVTFDWRSQNRAPAGIRP
SEQ ID NO 2: Human OSCAR-ECD
MALVLILQLLTLWPLCHTDITPSVPPASYHPKPWLGAQPATVVTPGVNVTLRCRAP
QPAWRFGLFKPGEIAPLLFRDVSSELAEFFLEEVTPAQGGSYRCCYRRPDWGPGVWSQPS
DVLELLVTEELPRPSLVALPGPVVGPGANVSLRCAGRLRNMSFVLYREGVAAPLQYRHSAQ
PWADFTLLGARAPGTYSCYYHTPSAPYVLSQRSEVLVISWEDSGSSDYTR
SEQ ID NO 3: Human OSCAR-S1a
MALVLILQLLTLWPLCHTDITPSVPPASYHPKPWLGAQPATVVTPGVNVTLRCRAP
QPAWRFGLFKPGEIAPLLFRDVSSELAEFFLEEVTPAQGGSYRCCYRRPDWGPGVWSQPS
DVLELLVTEELPRPSLVALPGPVVGPGANVSLRCAGRLRNMSFVLYREGVAAPLQYRHSAQ
PWADFTLLGARAPGTYSCYYHTPSAPYVLSQRSEVLVISWEGEGPEARPASSAPGMQAPG
PPPSDPGAQAPSLSSFRPRGLVLQPLLPQTQDSWDPAPPPSDPGV
SEQ ID NO 4: Human OSCAR-S1b
MALVLILQLLTLWPLCHTDITPSVAIIVPPASYHPKPWLGAQPATVVTPGVNVTLRC
RAPQPAWRFGLFKPGEIAPLLFRDVSSELAEFFLEEVTPAQGGSYRCCYRRPDWGPGVWS
QPSDVLELLVTEELPRPSLVALPGPVVGPGANVSLRCAGRLRNMSFVLYREGVAAPLQYRH
SAQPWADFTLLGARAPGTYSCYYHTPSAPYVLSQRSEVLVISWEGEGPEARPASSAPGMQ
APGPPPSDPGAQAPSLSSFRPRGLVLQPLLPQTQDSWDPAPPPSDPGV

WO2010040998 discloses that collagen proteins/peptides can act as ligands for OSCAR. WO2010040998 furthermore discloses that OSCAR binding to collagen peptides stimulates the activation and/or differentiation of OC. The effect can be blocked by the 11.1CN5 antibody. The 11.1CN5 antibody is an anti-human OSCAR mAb and it is an R-phycoerythrin conjugated antibody provided in a composition comprising sodium azide (https://www.beckmancoulter.com/wsrportal/search/Anti-OSCAR-PE/#2/10//0/25/1/0/asc/2/Anti-OSCAR-PE///0/1//0/).

Binding of antibodies and ligands to OSCAR can be measured by ELISA and SPR using recombinant proteins or by FACS and FMAT using transfected cells or cells naturally expressing OSCAR, such as e.g. monocytes. OSCAR activation upon ligand binding can be assessed by e.g. analysing expression of cell surface markers using FACS, by detecting cytokine release using ELISA or Bio-Plex; by detecting a reporter gene activity or by using an osteoclast formation assay.

Collagen: Collagen is the main component of connective tissue and extracellular matrix and are the most abundant protein in mammals. Collagen type I is mainly found in skin, tendon, vascular, ligature and bone (major protein component of bone). Collagen type II is mainly found in cartilage (major protein component of cartilage). Collagen type III is mainly found in reticulate (major component of reticular fibres). Collagen types I, II, III, and IV are the “fibrillar collagens”. Collagen I may herein be referred to as “ColI”, collagen II as “ColII, collagen III as “ColIII” and collagen IV as “ColIV”.

Therapeutic Applications: The compounds according to the invention can be used in the treatment of diseases involving an inappropriate or undesired immune response (immunological disorders), such as inflammation, autoimmunity, and conditions involving such mechanisms as well as graft vs. host disease. In one embodiment, such disease or disorder is an autoimmune and/or inflammatory disease. Examples of such autoimmune and/or inflammatory diseases are systemic lupus erythematosus (SLE), rheumatoid arthritis (RA), inflammatory bowel disease (IBD) (including ulcerative colitis (UC) and Crohn's disease (CD)), multiple sclerosis (MS), scleroderma, type 1 diabetes (T1 D), pemphigus vulgaris (PV), psoriasis, atopic dermatitis, celiac disease, Hashimoto's thyroiditis, Graves' disease, Sjogren's syndrome, Guillain-Barre syndrome, Goodpasture's syndrome, Addison's disease, Wegener's granulomatosis, primary biliary sclerosis, sclerosing cholangitis, autoimmune hepatitis, polymyalgia rheumatica, Raynaud's syndrome, temporal arteritis, giant cell arteritis, autoimmune hemolytic anemia, pernicious anemia, polyarteritis nodosa, Behcet's disease, primary bilary cirrhosis, uveitis, myocarditis, rheumatic fever, ankylosing spondylitis, glomerulonephritis, sarcoidosis, dermatomyositis, myasthenia gravis, polymyositis, alopecia greata, and vitilgo. The compounds according to the invention can furthermore be used in treatment of bone resorption related disorders such as e.g. osteoarthritis, osteoporosis, psoriatic arthritis, etc.

Rheumatoid arthritis (RA): RA is a systemic autoimmune disease that affects the entire body and is one of the most common forms of arthritis. It is characterized by the inflammation of the synovial membrane lining a joint, which causes pain, stiffness, warmth, redness and swelling. Inflammatory cells release enzymes that may digest bone and cartilage. As a result of RA, the inflamed joint lining, the synovium, can invade and damage bone and cartilage leading to joint deterioration and severe pain amongst other physiologic effects. The involved joint can lose its shape and alignment, resulting in pain and loss of movement. Anti-inflammatory biological therapeutics are now available for the treatment of rheumatoid arthritis (e.g. anti-CD20, a TNF-R antagonist or anti-TNF-α). However, none of these medicaments are specifically reducing bone erosion in RA patients.

There are several animal models for RA known in the art. For example, in the collagen-induced arthritis (CIA) model, mice develop chronic inflammatory arthritis that closely resembles human RA.

The term “treatment”, as used herein, refers to the medical therapy of any human subject or other animal subject in need thereof. Said subject is expected to have undergone physical examination by a medical or veterinary medical practitioner, who has given a tentative or definitive diagnosis which would indicate that the use of said specific treatment is beneficial to the health of said human or other animal subject. The timing and purpose of said treatment may vary from one individual to another, according to the status of the subject's health. Thus, said treatment may be prophylactic, palliative, symptomatic and/or curative. In terms of the present invention, prophylactic, palliative, symptomatic and/or curative treatments may represent separate aspects of the invention.

The term “antibody” herein refers to a protein, derived from a germline immunoglobulin sequence capable of specifically binding to an antigen or a portion thereof. The term includes full length antibodies of any isotype (i.e. IgA, IgD, IgE, IgG, IgM and/or IgY), any antigen binding fragment thereof, and any chain thereof. Examples of various types of molecules being encompassed by the definition of “antibodies” are listed below. Antibodies may be referred to in various ways herein, e.g. “antibody”, “Ab”, “mAb”, “monoclonal antibody”, etc, and such terms may be used interchangeably.

Full-length antibodies usually comprise at least four polypeptide chains: that is, two heavy (H) chains and two light (L) chains that are interconnected by disulfide bonds. One immunoglobulin sub-class of particular pharmaceutical interest is the IgG family, which may be sub-divided into isotypes IgG1, IgG2, IgG3 and IgG4. IgG molecules are composed of two heavy chains, usually interlinked by two or more disulfide bonds, and two light chains, ordinarily attached to a heavy chain by a disulfide bond. A heavy chain may comprise a heavy chain variable region (VH) and up to three heavy chain constant (CH) regions: CH1, CH2 and CH3. A light chain may comprise a light chain variable region (VL) and a light chain constant region (CL). VH and VL regions can be further subdivided into regions of hypervariability, termed complementarity determining regions (CDRs), interspersed with regions that are more conserved, termed framework regions (FR). VH and VL regions are typically composed of three CDRs and four FRs, arranged from amino-terminus to carboxy-terminus in the following order: FR1, CDR1, FR2, CDR2, FR3, CDR3, FR4. The hypervariable regions of the heavy and light chains form a binding domain that is capable of interacting with an antigen, whilst the constant region of an antibody may mediate binding of the immunoglobulin to host tissues or factors, including but not limited to various cells of the immune system (effector cells), Fc receptors and the first component (Clq) of the classical complement system.

Examples of antigen-binding fragments include Fab, Fab′, F(ab)2, F(ab′)2, F(ab)S, Fv (typically the VL and VH domains of a single arm of an antibody), single-chain Fv (scFv; see e.g. Bird et al., Science 1988; 242:42 S-426; and Huston et al. PNAS 1988; 85:5879-5883), dsFv, Fd (typically the VH and CH1 domain), and dAb (typically a VH domain) fragments; VH, VL, VhH, and V-NAR domains; monovalent molecules comprising a single VH and a single VL chain; minibodies, diabodies, triabodies, tetrabodies, and kappa bodies (see, e.g., Ill et al. Protein Eng 1997; 10:949-57); camel IgG; IgNAR; as well as one or more isolated CDRs or a functional paratope, where the isolated CDRs antigen-binding residues or polypeptides can be associated or linked together so as to form a functional antibody fragment. Various types of antibody fragments have been described or reviewed in, e.g., Holliger and Hudson, Nat Biotechnol 2005; 2S:1126-1136; WO2005040219, and published U.S. Patent Applications 20050238646 and 20020161201.

The term “antigen-binding fragment” of an antibody refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding fragment” of an antibody include a Fab fragment, a F(ab′)₂ fragment, a Fab′ fragment, a Fd fragment, a Fv fragment, a ScFv fragment, a dAb fragment and an isolated complementarity determining region (CDR). Single chain antibodies such as scFv and heavy chain antibodies such as VHH and camel antibodies are also encompassed within the term “antigen-binding portion” of an antibody. These antibody fragments may be obtained using conventional techniques known to those of skill in the art, and the fragments may be screened for utility in the same manner as intact antibodies.

“Fab fragments”, including “Fab” and “Fab(ab)₂” fragments, of an antibody are derived from said antibody by cleavage of the heavy chain in the hinge region on the N-terminal or C-terminal side of the hinge cysteine residues connecting the heavy chains of the antibody. A “Fab” fragment includes the variable and constant domains of the light chain and the variable domain and the first constant domain (CH1) of the heavy chain. “F(ab′)₂” fragments comprise a pair of “Fab” fragments that are generally covalently linked by their hinge cysteines. A Fab′ is usually derived from a F(ab′)₂ fragment by cleavage of the hinge disulfide bonds connecting the heavy chains in the F(ab′)₂. Other chemical couplings than disulfide linkages of antibody fragments are also known in the art. A Fab fragment retains the ability of the parent antibody to bind to its antigen. F(ab′)₂ fragments are capable of divalent binding, whereas Fab and Fab′ fragments can bind monovalently. Generally, Fab fragments lack the constant CH2 and CH3 domains, i.e. the Fc part, where interaction with the Fc receptors would occur. Thus, Fab fragments are in general devoid of effector functions. Fab fragments may be produced by methods known in the art, either by enzymatic cleavage of an antibody, e.g. using papain to obtain the Fab or pepsin to obtain the F(ab′) ₂, or Fab fragments may be produced recombinantly using techniques that are well known to the person skilled in the art.

An “Fv” fragment is an antibody fragment that contains an antigen recognition and binding site, and generally comprises a dimer of one heavy chain variable domain and one light chain variable domain. The association between the heavy chain variable domain and the light chain variable domain can be covalent in nature, for example in a single chain variable domain fragment (scFv). It is in this configuration that the three hypervariable regions of each variable domain interact to define an antigen-binding site/paratope on the surface of the VH-VL dimer. Collectively, the six hypervariable regions or a subset thereof confer antigen binding specificity to the antibody. However, even a single variable domain comprising only three hypervariable regions specific for an antigen has the ability to recognize and bind antigen, although usually at a lower affinity than the entire binding site (Cai & Garen, Proc. Natl. Acad. Sci. USA, 93: 6280-6285, 1996). For example, naturally occurring camelid antibodies that only have a heavy chain variable domain (VHH) can bind antigen (Desmyter et al., J. Biol. Chem., 277: 23645-23650, 2002; Bond et al., J. Mol. Biol. 2003; 332: 643-655).

“Single-chain Fv” or “scFv” antibody fragments comprise the VH and VL domains, where these domains are present in a single polypeptide chain. Generally, the Fv polypeptide further comprises a polypeptide linker between the VH and VL domains that enables the scFv to form the desired structure for antigen binding. For a review of scFv, see Pluckthun, 1994, In: The Pharmacology of Monoclonal Antibodies, Vol. 113, Rosenburg and Moore eds. Springer-Verlag, New York, pp. 269-315.

The term “diabodies” refers to small antibody fragments with two antigen-binding sites, in which fragments comprise a heavy chain variable domain (VH) connected to a light chain variable domain (VL) in the same polypeptide chain (VH and VL) by way of a linker. By using a linker that is too short to allow pairing between the two variable domains on the same chain, the variable domains are forced to pair with complementary domains of another chain, creating two antigen-binding sites. Diabodies are described more fully, for example, in EP 404,097; WO 93/11161; and Hollinger et al., 1993, Proc. Natl. Acad. Sci. USA, 90:6444-6448.

The expression “linear antibodies” refers to antibodies as described in Zapata et al., 1995, Protein Eng., 8(10):1057-1062. Briefly, these antibodies contain a pair of tandem Fd segments (VH-CH1-VH-CH1) that, together with complementary light chain polypeptides, form a pair of antigen binding regions. Linear antibodies can be bispecific or monospecific.

The term “monobody” as used herein, refers to an antigen binding molecule with a heavy chain variable domain and no light chain variable domain. A monobody can bind to an antigen in the absence of light chains and typically has three hypervariable regions, for example CDRs designated CDRH1, CDRH2, and CDRH3. An IgG monobody has two heavy chain antigen binding molecules connected by a disulfide bond. The heavy chain variable domain comprises one or more hypervariable regions, preferably a CDRH3 or HVL-H3 region.

Antibody fragments may be obtained using conventional recombinant or protein engineering techniques. Antibody fragments of the invention may be made by truncation, e.g. by removal of one or more amino acid residues from the N and/or C-terminal ends of a polypeptide. Fragments may also be generated by one or more internal deletions.

An antibody of the invention may be a human antibody or a humanized antibody. The term “human antibody”, as used herein, is intended to include antibodies having variable regions in which at least a portion of a framework region and/or at least a portion of a CDR region are derived from human germline immunoglobulin sequences. For example, a human antibody may have variable regions in which both the framework and CDR regions are derived from human germline immunoglobulin sequences. Furthermore, if the antibody contains a constant region, the constant region is also derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo).

Such a human antibody may be a human monoclonal antibody. Such a human monoclonal antibody may be produced by a hybridoma which includes a B cell obtained from a transgenic nonhuman animal, e.g., a transgenic mouse, having a genome comprising a human heavy chain transgene and a light chain transgene fused to an immortalized cell.

Human antibodies may be isolated from sequence libraries built on selections of human germline sequences, further diversified with natural and synthetic sequence diversity.

Human antibodies may be prepared by in vitro immunisation of human lymphocytes followed by transformation of the lymphocytes with Epstein-Barr virus.

The term “human antibody derivative” refers to any modified form of the human antibody, such as a conjugate of the antibody.

The term “humanized antibody”, as used herein, refers to a human/non-human chimeric antibody that contains a sequence (CDR regions) derived from a non-human immunoglobulin. A humanized antibody is, thus, a human immunoglobulin (recipient antibody) in which at least one or more residues from a hyper-variable region of the recipient are replaced by residues from a hypervariable region of a non-human species (donor antibody) for example from a mouse, rat, rabbit, or non-human primate, which have a desired property, such as specificity, affinity, and capacity. In some instances, FR residues of the human immunoglobulin are replaced by corresponding non-human residues. An example of such a modification is the introduction of one or more so-called back-mutations.

Furthermore, humanized antibodies may comprise residues that are not found in the recipient antibody or in the donor antibody. These modifications are made to further refine antibody performance. In general, a humanized antibody will comprise at least one—typically two—variable domains, in which all or substantially all of the CDR regions correspond to those of a non-human immunoglobulin and in which all or substantially all of the FR residues are those of a human immunoglobulin sequence. The humanized antibody can, optionally, also comprise at least a portion of an immunoglobulin constant region (Fc), typically that of a human immunoglobulin.

The term “humanized antibody derivative” refers to any modified form of the humanized antibody, such as a conjugate of the antibody and another agent or antibody.

The term “chimeric antibody”, as used herein, refers to an antibody whose light and heavy chain genes have been constructed, typically by genetic engineering, from immunoglobulin variable and constant region genes that originate from different species. For example, the variable segments of genes from a mouse monoclonal antibody may be joined to human constant segments.

The fragment crystallizable region (“Fc region”/“Fc domain”) of an antibody is the C-terminal region of an antibody, which comprises the constant CH2 and CH3 domains.

The Fc domain may interact with cell surface receptors called Fc receptors, as well as some proteins of the complement system. The Fc region enables antibodies to interact with the immune system. In one aspect of the invention, antibodies may be engineered to include modifications within the Fc region, typically to alter one or more of its functional properties (i.e. Fc effector functions), such as serum half-life, complement fixation, Fc-receptor binding, protein stability and/or antigen-dependent cellular cytotoxicity, or lack thereof, among others. Furthermore, an antibody of the invention may be chemically modified (e.g., one or more chemical moieties can be attached to the antibody) or may be modified to alter its glycosylation, so as to alter one or more functional properties of the antibody. Preferably, a modified Fc domain comprises one or more, and perhaps all of the following mutations that will result in decreased affinity to certain Fc receptors (L234A, L235E, and G237A) and in reduced Clq-mediated complement fixation (A330S and P331S), respectively (residue numbering according to the EU index). Antibodies having this type of Fc domain are sometimes referred to as “non-depleting” antibodies.

Antibodies without an Fc domain, such as e.g. therapeutic Fab fragments and other monovalent antibodies, tend to have a relatively short in vivo circulatory half life. In order to decrease the frequency of infusions with this type of therapeutic molecules, it may be desirable to conjugate such molecules with a “half life extending moiety”. Examples of half life extending moieties include: Biocompatible fatty acids and derivatives thereof, Hydroxy Alkyl Starch (HAS) e.g. Hydroxy Ethyl Starch (HES), Poly Ethylen Glycol (PEG), Poly (Glyx-Sery)n (HAP), Hyaluronic acid (HA), Heparosan polymers (HEP), Phosphorylcholine-based polymers (PC polymer), Fleximers, Dextran, Poly-sialic acids (PSA), an Fc domain, Transferrin, Albumin, Elastin like peptides, XTEN polymers, Albumin binding peptides, a CTP peptide, and any combination thereof.

The isotype of an antibody of the invention may be IgG, such as IgG1, such as IgG2, such as IgG4. If desired, the class of an antibody may be “switched” by known techniques. For example, an antibody that was originally produced as an IgM molecule may be class switched to an IgG antibody. Class switching techniques also may be used to convert one IgG subclass to another, for example: from IgG1 to IgG2 or IgG4; from IgG2 to IgG1 or IgG4; or from IgG4 to IgG1 or IgG2. Engineering of antibodies to generate constant region chimeric molecules, by combination of regions from different IgG subclasses, can also be performed.

In one embodiment, the hinge region of CH1 is modified such that the number of cysteine residues in the hinge region is altered, e.g., increased or decreased. This approach is described further for instance in U.S. Pat. No. 5,677,425 by Bodmer et al.

The constant region may further be modified to stabilize the antibody, e.g., to reduce the risk of a bivalent antibody separating into two monovalent VH-VL fragments. For example, in an IgG4 constant region, residue S241 may be mutated to a proline (P) residue to allow complete disulphide bridge formation at the hinge (see, e.g., Angal et al., Mol. Immunol. 199S; 30:105-8).

Antibodies or fragments thereof may also be defined in terms of their complementarity determining regions (CDRs). The term “complementarity determining region” or “hypervariable region”, when used herein, refers to the regions of an antibody in which amino acid residues involved in antigen binding are situated. The CDRs are generally comprised of amino acid residues 24-34 (L1), 50-56 (L2) and 89-97 (L3) in the light-chain variable domain and 31-35 (H1), 50-65 (H2) and 95-102 (H3) in the heavy-chain variable domain; (Kabat et al. (1991) Sequences of Proteins of Immunological Interest, Fifth Edition, U.S. Department of Health and Human Services, NIH Publication No. 91-3242) and/or those residues from a “hypervariable loop” (residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol. 1987; 196:901-917).

The term “antigen” (Ag) refers to the molecular entity used for immunization of an immunocompetent vertebrate to produce the antibody (Ab) that recognizes the Ag. Herein, Ag is termed more broadly and is generally intended to include target molecules that are specifically recognized by the Ab, thus including fragments or mimics of the molecule used in the immunization process, or other process, e.g. phage display, used for generating the Ab.

The term “epitope”, as used herein, is defined in the context of a molecular interaction between an “antigen binding polypeptide”, such as an antibody (Ab), and its corresponding antigen (Ag). Generally, “epitope” refers to the area or region on an Ag to which an Ab specifically binds, i.e. the area or region in physical contact with the Ab. Physical contact may be defined through various criteria (e.g. a distance cut-off of about 2-6 Å, such as 3 Å, such as 4 Å, such as 5 Å; or solvent accessibility) for atoms in the Ab and Ag molecules. A protein epitope may comprise amino acid residues in the Ag that are directly involved in binding to a Ab (also called the immunodominant component of the epitope) and other amino acid residues, which are not directly involved in binding, such as amino acid residues of the Ag which are effectively blocked by the Ab, i.e. amino acid residues within the “solvent-excluded surface” and/or the “footprint” of the Ab.

The epitope for a given Ab/Ag pair can be described and characterized at different levels of detail using a variety of experimental and computational epitope mapping methods. The experimental methods include mutagenesis, X-ray crystallography, Nuclear Magnetic Resonance (NMR) spectroscopy, Hydrogen deuterium eXchange Mass Spectrometry (HX-MS) and various competition binding methods as well as other methods that are known in the art. As each method relies on a unique principle, the description of an epitope is intimately linked to the method by which it has been determined. Thus, depending on the epitope mapping method employed, the epitope for a given Ab/Ag pair may be described differently.

At its most detailed level, the epitope for the interaction between the Ag and the Ab can be described by the spatial coordinates defining the atomic contacts present in the Ag-Ab interaction, as well as information about their relative contributions to the binding thermodynamics. At a less detailed level, the epitope can be characterized by the spatial coordinates defining the atomic contacts between the Ag and Ab. At an even less detailed level the epitope can be characterized by the amino acid residues that it comprises as defined by a specific criteria such as the distance between or solvent accessibility of atoms in the Ab-Ag complex. At a further less detailed level the epitope can be characterized through function, e.g. by competition binding with other Abs. The epitope can also be defined more generically as comprising amino acid residues for which substitution by another amino acid will alter the characteristics of the interaction between the Ab and Ag.

Epitopes described at the amino acid level, e.g. determined from an X-ray structure, are said to be identical if they contain the same set of amino acid residues. Epitopes are said to overlap if at least one amino acid is shared by the epitopes. Epitopes are said to be separate (unique) if no amino acid residue is shared by the epitopes.

The definition of the term “paratope” is derived from the above definition of “epitope” by reversing the perspective. Thus, the term “paratope” refers to the area or region on the Ab to which an Ag specifically binds, i.e. with which it makes physical contact to the Ag. Thus, a paratope may be defined by using the definition for epitope and applying it to the antibody as opposed to the antigen.

Antibodies that bind to the same antigen can be characterised with respect to their ability to bind to their common antigen simultaneously and may be subjected to “competition binding”/“binning”. In the present context, the term “binning” refers to a method of grouping antibodies that bind to the same antigen. “Binning” of antibodies may be based on competition binding of two antibodies to their common antigen in assays based on standard techniques such as surface plasmon resonance (SPR), ELISA or flow cytometry.

A “bin” can be viewed as an epitope defined using a reference antibody. If a second antibody is unable to bind to the antigen at the same time as the reference antibody, the second antibody is said to belong to the same “bin” as the reference antibody. In this case the reference and the second antibody are competing for binding to the antigen, thus the pair of antibodies is termed “competing antibodies”. If a second antibody is capable of binding to the antigen at the same time as the reference antibody, the second antibody is said to belong to a separate “bin”. In this case the reference and the second antibody are not competing for binding to the antigen, thus the pair of antibodies is termed “non-competing antibodies”.

Antibody “binning” does not provide direct information about the epitope. Competing antibodies, i.e. antibodies belonging to the same “bin” may have identical epitopes, overlapping epitopes or even separate epitopes. The latter is the case if the reference antibody bound to its epitope on the antigen takes up the space required for the second antibody to contact its epitope on the antigen (“steric hindrance”). Non-competing antibodies generally have separate epitopes.

“Reduction/Inhibition of the interaction of OSCAR with collagen” or “competing with collagen for binding to OSCAR” means the ability of an antibody, or a fragment thereof, to reduce in a dose dependent manner, the binding of collagen or fragments thereof, to OSCAR. A method to measure this can be, but is not limited to, surface plasmon resonance (SPR) analysis performed on a biosensor instrument, e.g. Biacore instruments as described in example 13 (50 nM OSCAR is used as an analyte and mixed with 0-10 μg/ml prior to injection into e.g. a Biocore T 100 instrument (GE healthcare) Human collagen I and collagen II are immobilized on e.g. a Biacore CM3 sensor chip (GE Healthcare)) OSCAR Antibodies according to the invention having the ability to significantly reduce OSCAR:collagen binding are those that can reduce OSCAR:collagen binding by at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95% compared to e.g. a control antibody.

Reduction of collagen induced cytokine secretion means the ability of an antibody, or a fragment thereof, to reduce in a dose dependent manner collagen induced cytokine secretion. Collagen, such as collagen I, can induce secretion of various cytokines (e.g. IL-8, TNF-α, etc.) by various cell types (e.g. synovial fluid mononuclear cells). Collagen may furthermore induce maturation of dendritic cells (DCs). Reduction of collagen induced cytokine secretion can e.g. be measured in the following way: Collagen I and collagen II are immobilized on a surface of essentially neutral charge (e.g. Costar #3361). RA synovial fluid cells (e.g. 3×10⁵/well) are preincubated with the anti OSCAR mAbs or control mAbs prior to be transferred onto the collagen coated plate. Cytokines are measured by ELISA. OSCAR antibodies according to the invention having the ability to reduce collagen induced cytokine secretion/DC cell maturation are those that can reduce cytokine secretion/DC cell maturation by at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95% compared to e.g. a control antibody.

Reduction of TRAP positive (TRAP⁺) osteoclast formation means the ability of an antibody, or a fragment thereof, to reduce in a dose dependent manner TRAP⁺ osteoclast formation. OSCAR antibodies according to the present invention may have the ability to reduce TRAP⁺ cell numbers in normal monocytes—and thereby reducing osteoclast formation. Reduction of TRAP⁺ osteoclast formation can e.g. be detected using the following assay: 60,000 monocytes/well are cultured with recombinant human M-CSF (25 ng/ml) and soluble RANKL (100 ng/ml). The cells are cultured on uncoated plastic. After ended incubation (day 9-12), the cells are stained for TRAP, e.g. using a leukocyte acid phosphatase kit. Compared to a control antibody, OSCAR antibodies according to the present invention may thus reduce TRAP⁺5 cell numbers in normal monocytes by at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%.

Reduction of TRAP5b secretion by osteoclasts means the ability of an antibody, or a fragment thereof, to reduce in a dose dependent manner TRAP5b secretion by osteoclasts. OSCAR antibodies according to the present invention may have the ability to reduce TRAP5b secretion by osteoclasts, wherein said osteoclasts are derived from normal monocytes. Reduction of TRAP5b secretion by osteoclasts can e.g. be detected using the following assay: 60,000 monocytes/well are cultured with recombinant human M-CSF (25 ng/ml) and soluble RANKL (100 ng/ml). The cells are cultured on uncoated plastic. After ended incubation (day 9-12), the supernatants from cell cultures are collected for ELISA measurements of TRAP5b. Compared to a control antibody, OSCAR antibodies according to the present invention may thus reduce TRAP5b secretion by osteoclasts derived from normal monocytes by at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%.

Reduction of bone resorption from osteologic discs and/or bone slices means the ability of an antibody, or a fragment thereof, to reduce in a dose dependent manner bone resorption from osteologic discs and/or bone slices. Reduction of bone resorption can e.g. be detected using the following assay: 60,000 monocytes/well are cultured with recombinant human M-CSF (25 ng/ml) and soluble RANKL (100 ng/ml). The cells are cultured on osteologic dics or bone slices. After ended incubation (day 9-12), bone resorption can be detected in one or more of the following way: (i) image analysis of erosion on osteologic discs/bone slides by e.g. Immunospot, and/or (ii) the supernatants are collected from the cell culture on bone slides and degraded collagen fragments are detected using e.g. CTX-1 ELISA. Compared to a control antibody, OSCAR antibodies according to the present invention may reduce bone resorption from osteologic discs and/or bone by at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%.

The terms “immunoreacts” or “immunoreacting”, as used herein, means any binding of an antibody to its epitope with a dissociation constant K_{d} lower than 10⁻⁴ M. The terms “immunoreacts” or “immunoreacting” are used where appropriate inter-changeably with the term “specifically bind”.

The term “affinity”, as used herein, means the strength of the binding of an antibody to an epitope. The affinity of an antibody is measured by the dissociation constant K_{d}, defined as [Ab]×[Ag]/[Ab−Ag] where [Ab−Ag] is the molar concentration of the antibody-antigen complex, [Ab] is the molar concentration of the unbound antibody and [Ag] is the molar concentration of the unbound antigen. The affinity constant Kₐ is defined by 1/K_{d}. Preferred methods for determining antibody specificity and affinity by competitive inhibition can be found in Harlow, et al., Antibodies: A Laboratory Manual, Cold Spring Harbor Laboratory Press, Cold Spring Harbor, N.Y., 1988), Colligan et al., eds., Current Protocols in Immunology, Greene Publishing Assoc. and Wiley Interscience, N.Y., (1992, 1993), and Muller, Meth. Enzymol. 92:589-601 (1983).

An “antagonist” is a compound that does not provoke a biological response upon binding to a receptor or a ligand, but blocks or dampens agonist-mediated responses. On the contrary, an “agonist” is a compound that induces a response upon binding to a receptor or ligand. Upon binding to a target, an antagonist will reduce the cell response induced by an agonist, such as e.g. a natural ligand or a compound that functionally resembles the effects of the natural ligand.

In connection with the present invention, the phrase “OSCAR induced effects” as used herein may refer to at least one of an inflammatory response or differentiation of OC. An inflammatory response can include but is not limited to activation of a cell of the immune system (e.g., T and B lymphocytes; polymorphonuclear leukocytes or granulocytes, such as neutrophils; monocytes, dendritic cells, macrophages, and NK cells), recruitment of a cell of the immune system, or release of a cell-derived soluble pro-inflammatory mediator, such as for example a cytokine or chemokine. Differentiation of OC can include, but is not limited to, expression of cellular markers for OC differentiation by monocytes-derived precursors or bone marrow precursors, increased cell fusion, increased tartrate-resistant acid phosphatase (TRAP) activity, increased cathepsin K expression, increased beta 3 integrin expression, multinucleation, and bone resorption. Consequently, a ligand can be said to antagonize OSCAR induced effects, for example, if it mediates at least about 20% inhibition, preferably at least about 30% inhibition, more preferably at least about 40% inhibition, more preferably at least about 50% inhibition, and most preferably at least about 75% inhibition of IL-8 release from dendritic cells (DCc) induced by ColI (example 8) at antibody concentrations below 10 nM.

A “receptor” is a protein molecule, embedded in either the plasma membrane (e.g. a transmembrane protein) or present in the cytoplasm of a cell, to which one or more specific signaling molecules may attach. A molecule which specifically binds (attaches) to a receptor is called a “ligand”, and may be an antibody, a peptide or other small molecule, such as a neurotransmitter, a hormone, a pharmaceutical drug, or a toxin. Ligand binding generally stabilizes a certain receptor conformation. This is often associated with gain of or loss of receptor activity, often leading to a cellular response. However, some ligands (e.g. antagonists) merely block receptors without inducing any response. Ligand-induced changes in receptors result in cellular changes which constitute the biological activity of the ligands.

Production of antibodies: Monoclonal antibodies are typically made by fusing myeloma cells with the spleen cells from a mouse that has been immunized with the desired antigen. Human antibodies can be obtained from transgenic animals (e.g. mice) encoding human antibodies. Alternatively, recombinant monoclonal antibodies can be made and identified involving technologies, referred to as repertoire cloning or phage display/yeast display. Recombinant antibody identificaion involves the use of viruses or yeast to display antibodies for selection, rather than the use of e.g. mice.

Antibodies may be produced by means of recombinant techniques. The DNA sequences encoding the antibody are usually inserted into a recombinant vector. The vector is preferably an expression vector in which the DNA sequence is operably linked to a promoter capable of directing the transcription of a cloned antibody gene or antibody cDNA in the desired host cell.

After the cells have taken up the DNA, they are grown in an appropriate growth medium, typically from a few days to a few weeks. The host cell may be any cell, which is capable of producing the antibody and includes bacteria, yeast, other fungi and higher eucaryotic cells. Examples of mammalian cell lines for use in the present invention are COS-1, baby hamster kidney (BHK) and HEK293. A preferred BHK cell line is the tk-ts13 BHK cell line that may be referred to as BHK 570 cells. In addition, a number of other cell lines, or their derivatives may be used within the present invention, including Rat Hep I, Rat Hep II, TCMK, NCTC 1469, CHO, and DUKX cells.

The transformed or transfected host cell described above is then cultured in a suitable nutrient medium under conditions permitting expression of the antibody. The antibody may be subsequently recovered from the culture by conventional procedures including separating the host cells from the medium by centrifugation or filtration, precipitating the proteinaceous components of the supernatant or its filtrate (for example, by means of a salt, e.g. ammonium sulphate), purification by a variety of chromatographic procedures, e.g. ion exchange chromatography, gelfiltration chromatography, affinity chromatography, or the like, dependent on the type of polypeptide in question.

Transgenic animal technology may be employed to produce the antibody of the invention. It is preferred to produce the antibodies in the mammary glands of a host female mammal, preferably in sheeps, goats or cattle. Production in transgenic plants may also be employed. Expression may be directed to a particular organ, such as a tuber.

The present invention further includes pharmaceutical compositions/formulations comprising antibodies according to the invention and optionally comprising a pharmaceutically acceptable carrier. The pharmaceutical composition according to the invention may be in the form of an aqueous formulation or a dried formulation that is reconstituted in water/an aqueous buffer prior to administration. Pharmaceutical compositions comprising compounds according to the invention may be supplied as a kit comprising a container that comprises the products according to the invention. Therapeutic polypeptides can be provided in the form of an injectable solution for single or multiple doses, or as a sterile powder that will be reconstituted before injection. Pharmaceutical compositions comprising compounds according to the invention are suitable for subcutaneous and/or IV administration.

### LIST OF EMBODIMENTS

The present invention is exemplified in the following non-limiting embodiments (it is understood that all embodiments can be combined):
1. An antibody that binds to human OSCAR and antagonizes OSCAR induced effects, wherein said antibody competes with antibody 4F3B for binding to OSCAR, wherein the amino acid sequence of a 4F3B heavy chain is as set forth in SEQ ID NO 18 and the amino acid sequence of a 4F3B light chain is as set forth in SEQ ID NO 17.
2. A ligand according to the present invention, wherein said ligand binds to a discontinuous epitope on OSCAR, wherein said epitope comprises at least one of amino acids 29-49 and at least one of amino acids 129-146 as set forth in SEQ ID NO 2.
3. An antibody according to the present invention, wherein said epitope comprises a discontinuous epitope, wherein said discontinuous epitope comprises amino acids 29-49 and 129-146 as set forth in SEQ ID NO 2.
4. An antibody comprising the three CDR sequences as set forth in SEQ ID NO 17 and three CDR sequences as set forth in SEQ ID NO 18. Preferably, said antibody comprises at least one CDR (e.g. CDR3) from SEQ ID NO 17 and at least one CDR (e.g. CDR3) from SEQ ID NO 18. One or two amino acid alterations (addition(s), and/or substitution(s) and/or deletion(s)) may be introduced into said CDR sequences. Said antibody may also comprise the entire VL/VH sequences set forth in SEQ ID NOs 17 and 18.
5. An antibody that binds to human OSCAR and antagonizes OSCAR induced effects, wherein said antibody competes with antibody 13F5A5 for binding to OSCAR, wherein the amino acid sequence of the 13F5A5 heavy chain is as set forth in SEQ ID NO 22, and the amino acid sequence of the 13F5A5 light chain is as set forth in SEQ ID NO 21.
6. An antibody according to the invention, wherein said antibody binds to the same epitope, or at least a portion of the same epitope, as the 13F5A5 antibody.
7. An antibody comprising the three CDR sequences as set forth in SEQ ID NO 21 and three CDR sequences as set forth in SEQ ID NO 22. Preferably, said antibody comprises at least one CDR (e.g. CDR3) from SEQ ID NO 21 and at least one CDR (e.g. CDR3) from SEQ ID NO 22. One or two amino acid alterations (addition(s), and/or substitution(s) and/or deletion(s)) may be introduced into said CDR sequences. Said antibody may also comprise the entire VL/VH sequences set forth in SEQ ID NOs 21 and 22
8. An antibody selected from the group consisting of: 4F3B, 13F5A5 (Hu), 13F56 (Hu), 16F119 (Hu), and 16F56 (Hu), wherein said antibody comprises at least the CDR3 CDR sequence of the 4F3B, 13F5A5 (Hu), 13F56 (Hu), 16F119 (Hu), and 16F56 (Hu) VH amino acid sequence and at least the CDR3 sequence of the 4F3B, 13F5A5 (Hu), 13F56 (Hu), 16F119 (Hu), and 16F56 (Hu) VL amino acid sequence. Preferably, said antibody comprises all three CDR sequences from the VH amino acid sequence (or the entire VH sequence) and all three CDR sequences of the VL amino acid sequence (or the entire VL sequence). 1-2 amino acids in one, two, three, four, five, or six of the CDR sequences may differ from 4F3B, 13F5A5 (Hu), 13F56 (Hu), 16F119 (Hu), and 16F56 (Hu) CDR sequences. Alternatively, said antibody comprises the entire VH/VL sequences of 4F3B, 13F5A5 (Hu), 13F56 (Hu), 16F119 (Hu), or 16F56 (Hu).
9. An antibody binding to the same epitope, or a portion thereof, of an antibody according to the invention.
10. An antibody according to the invention, wherein said antibody reduces interaction of OSCAR with collagen, wherein said collagen is selected from one or more from the group consisting of collagen I, collagen II, collagen III, and collagen IV, preferably collagen I and/or collagen II.
11. An antibody according to the invention, wherein said antibody competes with collagen I and/or collagen II for binding to human OSCAR.
12. An antibody according to the invention, wherein said antibody reduces collagen I induced IL-8 and/or TNF-α secretion by synovial fluid mononuclear cells from rheumatoid arthritis (RA) patients.
13. An antibody according to the invention, wherein said antibody reduces collagen I and/or collagen II induced TNF-α and/or IL-8 secretion by synovial fluid cells from Rheumatoid Arthritis (RA) patients.
14. An antibody according to the invention, wherein said antibody reduces osteoclast formation by inhibiting TRAP positive cell number in normal monocytes.
15. An antibody according to the invention, wherein said antibody reduces TRAP5b secretion by osteoclasts, wherein said osteoclasts are derived from normal monocytes.
16. An antibody according to the invention, wherein said antibody reduces bone resorption from osteologic discs and/or bone slices.
17. A pharmaceutical composition comprising an antibody according to the invention.
18. Use of an antibody according to the invention, or a pharmaceutical composition according to the invention, for treating an autoimmune inflammatory disease, such as e.g. RA.
19. Use of an antibody according to the invention, or a pharmaceutical composition according to the invention, for treating a bone resorption related disorder.
20. A method for inducing collagen dependent maturation of monocyte-derived Dendritic Cells (moDCs), wherein said method comprises incubating moDCs on a surface of essentially neutral charge in the presence of collagen.
21. A DNA molecule encoding an antibody according to the invention.
22. An expression vector comprising a DNA molecule according to the invention.
23. A host cell comprising an expression vector according to the invention and/or a DNA molecule according to the invention.
24. A method for making an antibody according to the invention, wherein said method comprises incubating a host cell according to the invention under conditions appropriate for expression of said antibody and optionally subsequently isolating said antibody.
25. Use of an antibody according to the invention, or a pharmaceutical composition according to the invention as a medicine.
26. A method of treatment comprising administering to a person in need thereof an appropriate amount of an antibody according to the invention for treatment of an autoimmune inflammatory disease, such as e.g. RA.
27. A method of treatment comprising administering to a person in need thereof an appropriate amount of an antibody according to the invention for treatment of a bone resorption related disorder, such as e.g. osteoarthritis, osteoporosis, psoriatic arthritis.
28. A method for inducing collagen dependent maturation of moDCs, wherein said method comprises incubating moDCs on a virgin polystyrene surface in the presence of collagen.
29. A method for inducing collagen dependent maturation of moDCs according to the invention, wherein collagen is selected from the group consisting of collagen I, collagen II, and collagen III.
30. Use of a method for inducing collagen dependent maturation of moDCs according to the invention for screening OSCAR antibodies for their ability to reduce maturation of moDcs.
31. An OSCAR variant comprising a C83A substitution.
32. Use of the OSCAR variant according to the invention for resolving OSCAR crystal structures.
33. Use of the OSCAR variant according to the invention for resolving the crystal structure of OSCAR bound to a ligand.

### EXAMPLES

### Example 1

### Production and Selection of OSCAR Antibodies

### Immunization and Fusion

In order to generate mouse monoclonal antibodies (mAb) BALB/c mice were immunized with the recombinant extracellular domain of human OSCAR (OSCAR-ECD (SEQ ID NO 2). Fully human mAbs were generated by immunization of transgenic mice (HuMab mice developed by Medarex Inc.) with OSCAR-ECD (SEQ ID NO 2). The HuMab mice have disabled murine IgG loci, and inserted loci encoding human IgG.

Mice were immunized subcutaneously. For the first immunization, 20 μg of antigen was mixed with complete Freunds adjuvant. In subsequent immunizations incomplete Freund's adjuvant was used with the same amount of antigen. Ten days after the last immunization, eye-blood from the mice was analyzed by ELISA for OSCAR specific antibodies. Mice with positive titers were boosted intravenously with 10 μg antigen in PBS, and sacrificed after 3 days. The spleens were aseptically removed and dispersed into a single cell suspension. Fusion of spleen cells and myeloma cells was performed using either the PEG-method or electrofusion. Hybridomas secreting specific antibodies were selected using specific binding assays as described below.

### Binding Assays: ELISA

Immunoplates (Maxisorb, Nunc) were coated with 2 μg/ml OSCAR-ECD. Hybridoma culture supernatants were added to the plates. Detection was carried out with a HRP-conjugated polyclonal antibody (pAb) specific to murine or human antibodies.

### Binding Assays: Flow Cytometry (FAGS)

Binding of antibodies to OSCAR was first examined in HEK293 cells transfected to express extracellular OSCAR. By using transfected cells it was possible to counter-screen for un-specific binding to the parental mock-transfected HEK293 cell line. The screening for OSCAR specific antibodies was done in sera, culture supernatants and in purified mAbs. Antibodies which selectively bound to the HEK-OSCAR cells were further tested for binding to primary monocytes from human PBMC expressing endogenous OSCAR.

This was done to ensure selection of antibodies which recognise OSCAR under physiological conditions.

### Establishment of the HEK-OSCAR Cell Line

Full length human OSCAR (hOSCAR) cDNA (GenBank #AF391162) was cloned into the NheI-BamHI sites of the mammalian expression vector pIRESneo2 (Clontech, #6938-1) and used for transfection of HEK293 cells. Transfected clones were selected for drug resistance (Geneticin, Invitrogen #10131-035). Expression of OSCAR was analysed by immunoblotting using anti-hOSCAR pAb (R&D, #AF2004). Cell surface expression of OSCAR was confirmed by FACS using the anti-OSCAR-PE (phycoerythrin) mAb clone 11.1CN5 (Beckman Coulter, #PN A24987). The HEK-hOSCAR clone 2-2D9 with the highest expression of OSCAR was selected for screening of anti-OSCAR mAbs. The geneticin-resistant pool of HEK293 cells transfected with pIRESneo2 (mock-transfectants) served as negative control.

### Purification of Primary Human Mononuclear Cells

Buffy coats from healthy individuals were obtained from the University Hospital of Copenhagen. The cells were diluted in RPMI 1640 and 25 ml put on top of 12.5 ml Ficoll-Paque Plus reagents (Amersham Biosciences) and spun down at 950×g for 30 min at room temperature. The peripheral blood mononuclear cells (PBMC) isolated by density gradient centrifugation were recovered from the interphase and thoroughly washed several times in RPMI 1640 medium, spun and re-suspended either in a washing buffer (1×PBS (—Ca²⁺/—Mg²⁺)+1% BSA) for immediate use or in a cryo-medium (RPMI 1640 supplemented with 20% FBS and 10% DMSO) for cryopreservation. Both freshly isolated and freeze-thawed cells were equally suitable for immunostaining followed by FACS analysis.

### Immunostaining and FACS Analysis

Cell number and viability were determined using a Cedex cell counter. The cells were diluted in washing buffer (1×PBS (—Ca²⁺/—Mg²⁺)+5% FBS+0.1% Na-azide) to 10⁷ cells/90 μl. In the case of monocytes, the cells were further blocked by adding 10 μl of a hFcR blocker (Miltenyi, #130-059-901) and incubated for 10 minutes at 4° C. 50 μl of cells (50.000 cells/well) were seeded into 96 well round-bottomed microtiter plates. Hybridoma supernatants or purified antibodies (in serial dilutions) were added to the cells at 50 μl/well. The isotype-matched purified irrelevant human mAb or mouse mAb served as negative controls (mouse IgG1, BD, #553452; mouse IgG2a, BD, #557353; mouse IgG2b, BD, #557352). The cells were incubated in the dark for 60 minutes at 4° C., washed twice and stained with the secondary antibodies (goat anti-human IgG-PE, Jackson ImmunoResearch, #109-116-170 or donkey anti-mouse IgG-APC (allophycocyanin), Jackson ImmunoResearch, #715-136-150), diluted 1:500, for 30 minutes in the dark at 4° C. Cells were washed in PBS, re-suspended in fixation buffer (1×PBS (—Ca²⁺/—Mg²⁺), pH7.2+1% formalin (B&B, #LAB96751.0500) and analysed by FACS flow cytometer. EC₅₀ values were calculated using a GraphPad Prism 5 software.

### Results and Conclusions

The majority of the mAbs which specifically bound to HEK-OSCAR cells were also capable of binding to primary human monocytes. The strongest binders were selected for further characterisation in functional assays. EC₅₀ values of the selected mAbs binding to human monocytes are shown in Table 1.

TABLE 1
Binding of anti-OSCAR mAbs to human monocytes

| mAb | EC₅₀, nM (Donor #1) | EC50, nM (Donor #2) |
|---|---|---|
| 7F1A1 | 1.21 | 0.12 |
| 7F6A1 | 3.48 | 0.14 |
| 7F14A1 | 2.47 | 0.55 |
| 7F21A1 | 9.48 | 0.01 |
| 7F10A1 | 1.2 | 0.12 |
| 13F5A5 | 4.02 | 2.18 |
| 13F56A6 | 1.54 | 2.39 |
| 3F7A9 | 1.79 | 0.14 |
| 4F3B | 0.66 | 0.32 |
| 1F7C7 | 4.63 | 3.68 |

### Example 2

### Determination of the Antigen Binding Affinity

Affinities of the anti-OSCAR mAbs towards OSCAR-ECD (SEQ ID NO 2) were measured by SPR using a Biacore T100 instrument (GE Healthcare). These studies were performed using either a direct binding procedure, with the respective mAb covalently coupled via free amine groups to the carboxy methylated dextrane membrane (CM5) on the sensor chip surface, or a capture method where the individual mAbs were captured on immobilized anti-mouse or anti-human pAbs prior to injection of OSACAR-ECD. OSCAR-ECD was injected in various concentrations, followed by a dissociation period with constant buffer flow over the sensor chip surface. The kinetic parameters (kₐ, k_{d} and K_{D}) for the interaction were calculated using a 1:1 interaction Langmuir fitting model.

### Results

The measured affinities were all in the nM range and comparable to that of the mAb 11.1CN5 which was shown to interfere with the OSCAR-ColI interaction. The affinity of OSCAR-ECD to ColI has been determined as 255 nM by SPR analysis. The sub-μM affinity of this interaction is within the range of the affinities reported for ColI interaction with other members of the LRC family, including LAIR-1/-2 and GPVI. Thus, the affinities of the mAbs to OSCAR are approximately 20-360 folds higher than the affinity of the OSCAR-ECD/ColI complex formation. Results are shown in Table 2.

TABLE 2
Affinities of OSCAR mAbs

| OSCAR | |
|---|---|
| mAbs | K_{D} (nM) |
| 3F7A9 | 9.1 |
| 4F3B | 1.7 |
| 7F1A1 | 2.7 |
| 7F6A1 | 2.7 |
| 7F10A1 | 18 |
| 7F21A1 | 18 |
| 7F14A1 | 4.5 |
| 13F5A5 | 0.7 |
| 13F56A6 | 3.0 |
| 16F56 | 0.3 |
| 16F119 | 0.5 |
| 11.1CN5 | 6.4 |

### Example 3

Cloning and Sequencing of the Anti-OSCAR mAb cDNAs

The cDNAs of the light and heavy chains were cloned by RT-PCR using total RNA extracted from 10⁷ cells of each anti-hOSCAR hybridomas 4F3B, 7F1A, 7F6A, 7F9A, 7F10A, 7F14A, 7F21A, and 3F7A9. Reverse transcription was performed using oligo-dT primer. Antibody cDNAs were amplified using specific primers. The amplified products were cloned into the pMD19-T vector (Takara, China) and sequenced. The cDNAs were then sub-cloned into a vector for expression in mammalian cells. The recombinant antibody were produced in HEK2936E cells and purified.

Amino acid sequences of the selected mAbs are listed below.

### 7F1A1 Light Chain and Heavy Chain Sequences (CDR Sequences Shown in Bold/Underline and VL/VH Sequences are Shown in Capital Letters):

7F1A1 Light Chain
(SEQ ID NO 5)
mklpyrllvllwltdarcDIQMTQSPASLSVSVGETVTITCRASEKIYSNLAWYQQKQGKSPQLLVYA
ATNLADGVPSRFSGSGSGTQFSLKINSLQPEDFGSYYCQHFWGIPWTFGGGTKLEIKRada
aptysifppsseqltsggasvvcflnnfypkdinvkwkidgserqngvlnswtdqdskdstysmsstlt
ltkdeyerhnsytceathktstspivksfnrnec
7F1A1 Heavy Chain
(SEQ ID NO 6)
mgwswvflfllsvtagvhsQVQLQQSGAELVRPGTSVKVSCKASGYAFTNYMIEWVKKRPGQGLE
WIGVINPGSGGTNYNEKFENKATLTADKSSSTAYMQLSSLTSDDSAVYFCARSLDFDGAWF
AYWGQGTLVTVSAakttppsvyplapgsaaqtnsmvtlgclvkgyfpepvtvtwnsgslssgvht
fpavlqsdlytlsssvtvpsstwpsetvtcnvahpasstkvdkkivprdc

### 7F6A1 Light Chain and Heavy Chain Sequences (CDR Sequences Shown in Bold/Underline and VL/VH Sequences are Shown in Capital Letters):

7F6A1 Light Chain
(SEQ ID NO 7)
mdlqvqiisfllisasvimsrgENVLTQSPAIMSASPGEKVTMTCSVSSSISYMHWYRQKSFTSPQLWI
YDTSKLASGVPGRFSGSGSGISYSLTISSMEAEDVATYYCFQGSEYPLTFGAGTKLELKRad
aaptvsifppsseqltsggasvvcflnnfypkdinvkwkidgserqngvlnswtdqdskdstysmsstl
tltkdeyerhnsytceathktstspivksfnrnec
7F6A1 Heavy chain
(SEQ ID NO 8)
mdfglsliflvltlkgvqcDVKLVESGGGLVKPGGSLKLSCAASGFTFSSYVMSWVRQTPEKRLEWV
ATISSGGSYTYYPDSVKGRFTISRDNAKNTLYLQMSSLKSEDTAMYYCTREGGNSPDYWG
QGTTLTVSSakttppsvyplapgsaaqtnsmvtlgclvkgyfpepvtvtwnsgslssgvhtfpavlqs
dlytlsssvtvpsstwpsetvtcnvahpasstkvdkkivprdc

### 7F9A1 Light Chain and Heavy Chain Sequences (CDR Sequences Shown in Bold/Underline and VL/VH Sequences are Shown in Capital Letters):

7F9A1 Light Chain
(SEQ ID NO 9)
metdtlllwvlllwvpgstgDIVLTQSPASLAVSLGQRATISCRASQSVSTSNSSYMHWCQQKPGQPP
KLLIKYASNLESGVPARFSGSGSGTDFTLNIHPVEEEDTATYYCQHSWEIPFTFGGGTKLEIK
Radaaptvsifppsseqltsggasvvcflnnfypkdinvkwkidgserqngvlnswtdqdskdstysm
sstltltkdeyerhnsytceathktstspivksfnrnec
7F9A1 Heavy Chain
(SEQ ID NO 10)
mewswvfifllsgtagvlsEVQLQQFGAELVKPGASVKISCKASGYTFTDYNMDWVKQSHGKSLEW
IGDINPNYDSTTYNQKFKGKATLTVDKSSSTAYMELRSLTSEDTAVYYCARAYGNYDWWYF
DVWGAGTTVTVSSakttppsvyplapgsaaqtnsmvtlgclvkgyfpepvtvtwnsgslssgvhtfp
avlqsdlytlsssvtvpsstwpsetvtcnvahpasstkvdkkivprdc

### 7F10A1 Light Chain and Heavy Chain Sequences (CDR Sequences Shown in Bold/Underline and VL/VH Sequences are Shown in Capital Letters):

7F10A1 Light Chain
(SEQ ID NO 11)
mdfqvgifsfllisasVIMSRGQILLTQSPAIMSASPGEKVTMTCSASSSVSYMHWYQQKPGSSPKP
WIYDTSNLASGFPARFSGSGSGTSYSLIISSMEAEDAATYYCHQRSSYPYTFGGGTKLEIKR
adaaptvsifppsseqltsggasvvcflnnfypkdinvkwkidgserqngvlnswtdqdskdstysms
stltltkdeyerhnsytceathktstspivksfnrnec
7F10A1 Heavy Chain
(SEQ ID NO 12)
mgwswvflfilsgtagvhsQVQLQQSGAELARPGASVKLSCKASGYTFTDNYINWVKQRTGQGLE
WIGEIYPGSGNTYYNEKFKGKATLTADKSSSTAYMQLSSLTSEDSAVYFCARSANRYDRGD
FDYWGQGTPLTVSSakttppsvyplapgsaaqtnsmvtlgclvkgyfpepvtvtwnsgslssgvhtfp
avlqsdlytlsssvtvpsstwpsetvtcnvahpasstkvdkkivprdc

### 7F14A1 Light Chain and Heavy Chain Sequences (CDR Sequences Shown in Bold/Underline and VL/VH Sequences are Shown in Capital Letters):

7F14A1 Light Chain
(SEQ ID NO 13)
mdfqvqiisfllisasviisrgQIVLTQSPAIMSASPGEKVTMTCSASSSVNYMHWYQQKSGTSPKRWI
YDTSNLASGVPTRFSGSGSGTSYSLTISSMEAEDAATYYCQQWSSDPLTFGAGTKLELKRa
daaptvsifppsseqltsggasvvcflnnfypkdinvkwkidgserqngvlnswtdqdskdstysmsstl
tltkdeyerhnsytceathktstspivksfnrnec
7F14A1 Heavy Chain
(SEQ ID NO 14)
*rlwlvlgllfclvafpscvlsQVQLKESGPGLVAPSQSLSITCSVSGFSLTSYGVHWVRQPPGKGLEW
LGVMWAGGSTNYNSALMSRLSISKDNSKSQVFLKMNSLQTDDSAMYYCALQKGYGNYFD
YWGQGTTLTVSSakttppsvyplapgsaaqtnsmvtlgclvkgyfpepvtvtwnsgslssgvhtfpav
lqsdlytlsssvtvpsstwpsetvtcnvahpasstkvdkkivprdc

### 7F21A1 Light Chain and Heavy Chain Sequences (CDR Sequences Shown in Bold/Underline and VL/VH Sequences are Shown in Capital Letters):

7F21A1 Light Chain
(SEQ ID NO 15)
msvltqvlgllllwltdarcDIQMTQSPASLSVSVGETVTITCRASEKIYSNLAWYQQKQGKSPOLLVY
AATNLADGVPSRFSGSGSGTQFSLKINNLQSEDFGSYYCQHFWGTPWTFGGGTKLEIKRa
daaptvsifppsseqltsggasvvcflnnfypkdinvkwkidgserqngvlnswtdqdskdstysmsst
ltltkdeyerhnsytceathktstspivksfnrnec
7F21A1 Heavy Chain
(SEQ ID NO 16)
mgwswvflfllsvtagvhsQVQLQQSGAELVRPGTSVKVSCRSSGYAFTNYLIEWIKQRPGQGLEW
IGVITPGSGGTNYNENFKDKATLTADKSSNTAYMQLTSLTSDDSAVYFCARSFDYDGAWFA
YWGQGTLVTVSAakttppsvyplapgsaaqtnsmvtlgclvkgyfpepvtvtwnsgslssgvhtfpav
lqsdlytlsssvtvpsstwpsetvtcnvahpasstkvdkkivprdc

### 4F3B Light Chain and Heavy Chain Sequences (CDR Sequences Shown in Bold/Underline and VL/VH Sequences are Shown in Capital Letters):

4F3B Light Chain
(SEQ ID NO 17)
mesqilvlmsllfwvsgtcgDIVMTQSPSSLTVTAGEKVTMSCKSSQSLLNSGNQKNYLTWYQQKP
GQPPKVLIYWASTRESGVPDRFTGSGSGTDFTLTISSVQAEDLAVYYCQNDYSYPLTFGTG
TKLELKRadaaptvsifppsseqltsggasvvcflnnfypkdinvkwkidgserqngvlnswtdqds
kdstysmsstltltkdeyerhnsytceathktstspivksfnrnec
4F3B Heavy Chain
(SEQ ID NO 18)
mgwsgififlfsvtagvhsQVQLQQSGAELAKPGASVKMSCKASGFTFTSYWMHWVKQRPGQGLE
WIGYINPSTDYTEYNQKFKDKATLTADKSSSTAYMQLSSLTSEDSAVYYCARRSPFAMDYW
GQGTSVTVSSakttppsvyplapgsaaqtnsmvtlgclykgyfpepvtvtwnsgslssgvhtfpav
lqsdlytlsssvtvpsstwpsetvtcnvahpasstkvdkkivprdc

### 3F7A9-1 Light Chain and Heavy Chain Sequences (CDR Sequences Shown in Bold/Underline and VL/VH Sequences are Shown in Capital Letters):

3F7A9-1 Light Chain
(SEQ ID NO 19)
mrapaqilgilllwfpgikcDIKMTQSPSSMYASLGERVTITCKASQDINSFLSWLQQKPGKSPKTLIY
RANRLVDGVPSRFSGSGSGQDYSLTISSLEYEDMGIYYCLQYDDFPYTFGGGTKLEIKRada
aptysifppsseqltsggasvvcflnnfypkdinvkwkidgserqngvlnswtdqdskdstysmsstlt
ltkdeyerhnsytceathktstspivksfnrnec
3F7A9-1 Heavy Chain
(SEQ ID NO 20)
mgwsgvflfllsgttgvhsEIQLQQSGPELVKPGASVKVSCKASGYVFTSYNMYWVKQSHGKSLEWI
GYIDPYNGGTRYNQKFKGKATLTVDKSSSTAYMHLNSLTSEDSAVYYCARLDYYGNFYWG
QGTTLTVSSakttppsvyplapgsaaqtnsmvtlgclvkgyfpepvtvtwnsgslssgvhtfpavlqsd
lytlsssvtvpsstwpsetvtcnvahpasstkvdkkivprdc

### 13F5A5 Variable Light Chain and Variable Heavy Chain Sequences (CDR Sequences Shown in Bold/Underline and VL/VH Sequences are Shown in Capital Letters):

13F5A5 Variable Light Chain
(SEQ ID NO 21)
EIVLTQSPGTLSLSPGERATLSCRASQSVSSSYLAWYQQKPGQAPRLLIYGASSRATGIPDR
FSGSGSGTDFTLTISRLEPEDFAVYYCQQYGSSPLTFGGGTKVEIKR
13F5A5 Variable Heavy Chain
(SEQ ID NO 22)
EVQLVQSGAEVKKPGESLKISCKGSGYSFTSYWIVWVRQMPGKGLEWMGSIYPGDSDARY
SPSFQGQVTISADKSISTAYLQWSSLKASDTAMYYCARQKITMVRGVIITPLDGMDVWGQG
TTVTVSS

### 13F56 Variable Light Chain and Variable Heavy Chain Sequences (CDR Sequences Shown in Bold/Underline and VL/VH Sequences are Shown in Capital Letters):

13F56 Variable Light Chain
(SEQ ID NO 23)
EIVLTQSPGTLSLSPGERATLSCRASQSVSSSYLAWYQQKPGQAPRLLIYGASSRATGIPDR
FSGSGSGTDFTLTISRLEPEDFAVYYCQQYGSSPTFGPGTKVDIKR
13F56 Variable Heavy Chain
(SEQ ID NO 24)
QVQLVQSGAEVKKPGSSVKVSCKASGGTFSSYAISWVRQAPGQGLEWMGKIIPILGITNYA
QKFQGRVTITADKSTSTAYMELSSLRSEDTAVYYCANPLTFDAFDIWGQGTMVTVSS

### 16F56 Variable Light Chain and Variable Heavy Chain Sequences (CDR Sequences Shown in Bold/Underline):

16F56 Variable Light Chain
(SEQ ID NO 25)
DIQMTQSPSFLSASVGDRVTITCRASQSISSYLNWYQQKPGKAPKLLIYAASSLQSGVPSRF
SGSGSGTDFTLTISSLQPEDFATYYCQCGYSTPLTFGGGTKVEIKR
16F56 Variable Heavy Chain
(SEQ ID NO 26)
QVQLVQSGAEVKKPGSSVKVSCKASGGTFSNYAISWVRQAPGQGLEWMGRIIPILGIAHNA
QKFQDRVTITADKSTSTAYMELSSLRSEDTAVYYCASPSSGKGLPYWFFDLWGRGTLVTVSS

### 16F119 Variable Light Chain and Variable Heavy Chain Sequences (CDR Sequences Shown in Bold/Underline):

16F119 Variable Light Chain
(SEQ ID NO 27)
EIVLTQSPGTLSLSPGERATLSCRASQSVSSSYLAWYQQKPGQAPRLLIYGASSRATGIPDR
FSGSGSGTDFTLTISRLEPEDFAVYYCQQYGSSLTFGGGTKVEIKR
16F56 Variable Heavy Chain
(SEQ ID NO 28)
QVQLVQSGAEVKKPGSSVKVSCKVSGGTFSNYAISWVRQAPGQGLEWMGRIIPILGIAAYA
QRFRDRVTITADKFTSTVYMELSSLRSEDTAVYYCASGQLGTPYWYFDLWGRGTLVTVSS

SEQ ID NO 29: 6F3A Variable Light Chain - CDR sequences shown in
bold/underline (the hybridomas 6F3A and 6F6B are identical clones):
DIVLTQSPATLSVTPGDSVSLSCRASQSITNNLHWYQQKSHESPRLLIKYASQSISGIPSRFR
GSGSGTDFTLSINSVETEDFGVYFCQQSNTWPRTFGGGTKLEIKR
SEQ ID NO 30: 6F3A Variable Heavy Chain - CDR sequences shown in
bold/underline (the hybridomas 6F3A and 6F6B are identical clones):
EVQLQQSGAELVKPGASVKLSCTASGFNIKDTYVHWVKQRPDQGLEWIGRIDPAIGNTKYD
PKFRGKATITADSSSNTAYLQLSSLTSEDTAVYYCARLSYGNYGLDYSMDYWGQGTSVTVSS

### Example 4

### SPR Analysis of Competition Between Anti-OSCAR mAb and ColI for Binding to OSCAR

The experiment was performed using a Biacore T 100 instrument (GE Healthcare). Human ColI (Sigma, # C5483) was diluted with 10 mM NaOAc, pH 4.5 to 10 μg/ml and immobilized on a Biacore CM3 sensor chip (GE Healthcare) using amine coupling chemistry (GE Healthcare). Immobilization was performed according to the standard procedure, but at 4° C. in order to prevent aggregation of ColI, with immobilized levels of ca 150 RU on flow cell (FC) 2 and ca 300 RU on FC 4. A blank immobilization was performed on FC 1 & 3, which were used as reference cells. HBS-EP buffer (10 mM HEPES, 150 mM NaCl, 3 mM EDTA, 0.005% Polysorbat P20, pH7.4) was used as running buffer for the immobilization. Buffer containing HBS-EP/+0.1% BSA was used for the competition assay.

The competition experiment was performed at 4° C. at a flow rate of 50 μl/min. The OSCAR-ECD and OSCAR-S1a proteins (SEQ ID NO 2 and SEQ ID NO 3, respectively) were used as analytes at a concentration of 50 nM. OSCAR was mixed with various concentrations of anti-OSCAR antibodies ranging from 0-5 μg/ml prior to injection. Binding curves were measured for 240 s followed by 200 s of dissociation. The chip surface was regenerated for 2×20 s with 10 mM glycine, pH 2.0 between assays. All assays were performed in duplicates.

### Results

Human ColI was immobilized to the surface of a Biacore sensor chip by amine coupling. Binding of OSCAR-ECD (SEQ ID NO 2) and OSCAR-S1a (SEQ ID NO 3) and binding of the soluble extracellular domain (ECD) of OSCAR to ColI was studied in the presence or absence of anti-OSCAR antibodies. Two different constructs of OSCAR were analysed: OSCAR-ECD (SEQ ID NO 2) and OSCAR-S1a (SEQ ID NO 3). It was hypothesized that an antibody inhibiting binding of OSCAR to ColI would lead to a concentration-dependent loss of signal, whereas a non-inhibiting antibody would enhance the signal due to the increased mass of the OSCAR-antibody complex. Of the 5 tested antibodies, 3 fell into one of the two groups. As shown in Table 3, mAbs 13F5A5 and 4F3B inhibited binding of OSCAR to ColI with very little binding left at 5 μg/ml antibody. In contrast, 3F7A9 did not interfere with binding since a concentration-dependent increase of the signal was detected.

Further SPR analysis of competition between anti-OSCAR mAbs and ColI/ColII for binding to OSCAR was done as described in Example 13.

TABLE 3
SPR analysis of the binding of OSCAR-ECD or
OSCAR-S1a to ColI immobilized at two densities

| | 150 RU colI | | 300 RU colI | |
|---|---|---|---|---|
| | Binding | Binding | Binding | Binding |
| | [RU] | [RU] | [RU] | [RU] |
| sample | no mAb | 5 μg mAb | no mAb | 5 μg mAb |
| OSCAR 1Sa/13F5A5 | 11.2 | −0.2 | 22.7 | 2.0 |
| OSCAR 1Sa/1F5H10-2 | 11.1 | 96.6 | 22.6 | 158.7 |
| OSCAR 1Sa/2F9G11 | 11.0 | 16.5 | 22.6 | 28.2 |
| OSCAR 1Sa/3F7A9 | 10.8 | 58.9 | 22.6 | 92.6 |
| OSCAR 1Sa/4F3B | 11.0 | −0.3 | 22.5 | 1.4 |
| OSCAR ECD/13F5A5 | 11.0 | 1.4 | 24.8 | 5.1 |
| OSCAR ECD/1F5H10-2 | 11.0 | 22.8 | 25.0 | 36.7 |
| OSCAR ECD/2F9G11 | 11.1 | 133.4 | 25.0 | 209.9 |
| OSCAR ECD/3F7A9 | 11.3 | 82.5 | 24.8 | 130.2 |
| OSCAR ECD/4F3B | 10.9 | 0.9 | 25.0 | 3.6 |

### Example 5

### Epitope Diversity Assessed by SPR Analysis

Antibody binding competition assays were performed by SPR analysis in order to investigate whether the anti-OSCAR mAbs bound simultaneously to OSCAR-ECD (SEQ ID NO 2) or cell-surface OSCAR on human PBMC. An inability of mAbs to bind simultaneously indicates common or overlapping epitopes, though factors such as steric hindrance and conformational changes may contribute.

SPR studies were performed using a BiacoreT100 instrument (GE Healthcare). The purified anti-OSCAR mAbs were immobilized on a CM5 sensor chip in individual flow cells. A fixed concentration of OSCAR-ECD in HBS-EP buffer (10 mM HEPES, 150 mM NaCl, 3 mM EDTA and 0.005% Polysorbat P20, pH7.4) was injected prior to injection of a second anti-OSCAR mAb. All mAbs were tested in all combinations.

### Results

Antibodies demonstrating an inhibitory effect on ColI-dependent dendritic cell maturation (described in Example 8) were represented in four bins/competitive groups (Table 4). This data demonstrates that interference with at least four distinct regions in OSCAR will influence the binding and/or activation by ColI. The ligand competing data (described in Example 13) differentiate the mAbs between the four bins.

TABLE 4
Binning of mAbs based on simultaneous binding to OSCAR

| Bin#1 | Bin# 2 | Bin# 3 | Bin# 4 |
|---|---|---|---|
| 4F3B | 11.1CN5 | 7F10A1 | 7F6A1 |
| 13F5A5 (Hu) | 3F7A9 | 7F14A1 | 6F3A |
| 13F56 (Hu) | 7F1A1 | | 6F6B |
| 16F119 (Hu) | 7F21A | | |
| 16F56 (Hu) | | | |

The mAbs in each bin/competitive group were not able to bind simultaneously to OSCAR.

### Example 6

### Epitope Diversity Assessed by Flow Cytometry Analysis

The aim of the binning study was to group the antibodies into competitive groups/bins according to their ability to inhibit binding of a fluorochrome labelled anti-OSCAR antibody to human monocytes. Inhibition of binding indicates that the two antibodies tested show steric hindrance of simultaneous binding to the target cells and hence belonging to the same bin. An inhibition of binding can be due to binding to the same epitope, binding to overlapping epitopes or binding to two non-overlapping epitopes albeit with steric hindrance of simultaneous binding.

All antibodies shown to inhibit cytokine release by a DC cytokine release assay (described in example 8) were analysed in the binning study.

### Antibodies

Labelling of mAbs was performed using the Alexa Fluor® 647 Monoclonal Antibody Labeling Kit (Molecular Probes, #A-20186) according to manufactures protocol. The following mouse anti-OSCAR mAbs were labelled: 4F3B, 3F7A9, 6F3A, 6F6B, 7F1A1, 7F6A1, 7F10A1, 7F12A1, 7F13A1, 7F14A1, 7F21A1 and the mAb 11.1CN5. The following human anti-OSCAR mAbs were labelled: 13F56A6, 13F5A5, 16F56A2 and 16F119A5. The same sets of antibodies were kept unlabelled.

For the quality control and facilitating gating of relevant cell populations, the following mAbs have been used: 7AAD (BD, #51-68981E), anti-CD3-FITC (BD, #345763), anti-CD19-FITC (BD, #345776) and anti-CD14-FITC (BD, #345784).

### Binding Assay

PBMC from healthy individuals were purified as described in Example 1. The cells were re-suspended in washing buffer (1×PBS (—Ca²⁺/—Mg²⁺)+1% BSA). Viability and cell numbers were calculated using a Cedex cell counter. 10⁷ cells (90 μl) were treated with 10 μl hFcR blocker (Miltenyi, #130-059-901). The cells were incubated 10 minutes at 4° C., followed by a dilution in washing buffer to 10⁶ cells/ml. Labelled antibodies were diluted in washing buffer to 2 μg/ml and added as 25 μl/well in a round bottom 96-well plategiving a final concentration of 0.5 ug/ml og labelled antibody. 25 μl of unlabelled antibody in 3-fold serial dilutions ranging from 15 ug/ml to 250 ng/ml (final concentration) were added to the same wells (according to plate outline). Then PBMC were added to all wells at 50 μl/well (50.000 cells/well). The cells were incubated in the dark at 4° C. for 60 minutes and washed twice in 200 μl washing buffer/well. Finally, the cells were washed once in PBS, re-suspended in 100 μl PBS/well and fixed with an equal volume of fixation buffer (1×PBS (—Ca²⁺/—Mg²⁺)+1% Formalin). The cells were kept in the dark at 4° C. until analysis by flow cytometry.

### Results and Conclusions

Consistent with the epitope binning results by SPR analysis (described in Example 5), the data from flow cytometry analysis on monocytes (CD14⁺ cells) showed that the inhibitory mAbs identified in a DC cytokine secretion assay were represented in four bins/competitive groups (Table 5).

The median fluorescence intensity was measured in the presence of various concentrations of unlabelled mAb and used to generate an inhibition curve. The results are shown in Table 5. Full inhibition (=at least about 75% inhibition, preferably at least about 90% inhibition, and most preferably at least about 95% inhibition) is shown as grey squares, while partial inhibition (=about 10-50% inhibition, or about 20-40% inhibition), or shown as dotted squares. No inhibition was seen with combinations of the antibodies indicated as white squares. For one of the antibodies, 7F6A1, it was difficult to definitely group it together with the other antibodies tested. Unlabelled 7F6A1 was fully inhibiting binding of labelled 4F3B, while unlabelled 4F3B only partially inhibited binding of the labelled 7F6A1. Binding of the four labelled human antibodies 13F5A5, 13F56A6, 16F56A2 and 16F119A5 was partially inhibited by F6A1 and 13F5A5 was also able to partially inhibit 7F6A1 which none of the other three human mAbs were able to. Briefly, The mAbs could be grouped into four main groups listed in Table 5. This main grouping is based exclusively on the full inhibition profile. Although it was not possible to label the 6F3A, this mAb has been groped together with 6F6B and 7F6A1. The partial inhibition profile is less clear. Both mAbs in a test were added simultaneously, there should be no preference for one of the antibodies compared to the other. Presumably, labelling of the antibodies with AlexaFlour 647 might have an effect on binding properties. There might be a change in affinity due to labelling of the antibody, or the flexibility of the F(ab) arms may be affected by the labelling.

TABLE 5
Binning of the mAbs by flow cytometry analysis

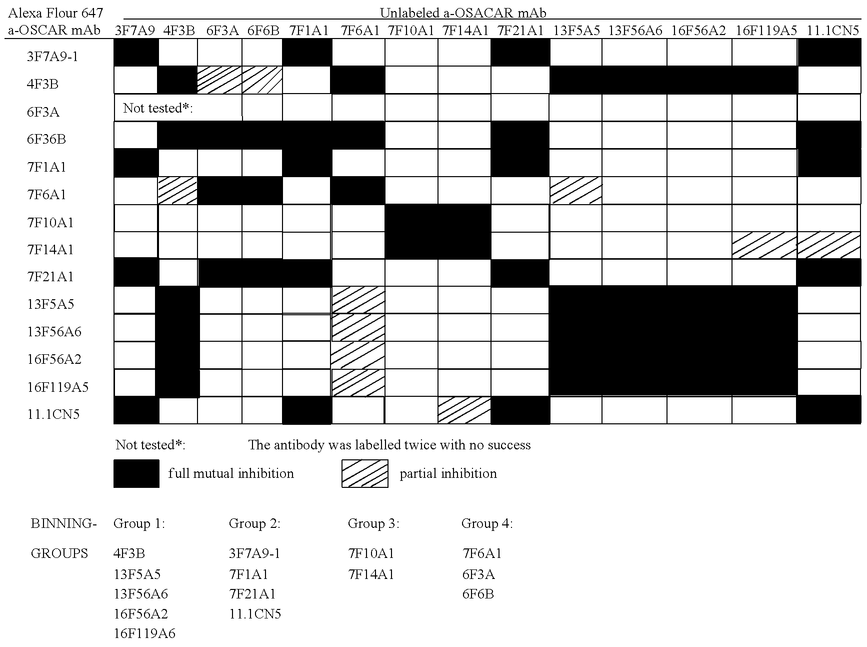

Black squares indicate full inhibition and the dashed squares indicate partial inhibition.

### Example 7

### Epitope Mapping by Peptide Array

A preliminary epitope prediction of the anti-OSCAR mAb 4F3B was done using a peptide walking technique. A peptide array was synthesised based on SEQ ID NO 2 with C to A mutations for all cysteins. A 20- (20 mer), 16- and 12 amino acid residue peptide walk was used in the design of the peptide array. The frame walking was set to 2 residues. The array was screened using the fluorescein-labelled mAb 4F3B.

### Synthesis of Epitope Arrays

The epitope mapping arrays were synthesized on cellulose sheets (Aims-Scientific) using an array synthesizer (Multipep Spot, Intavis) according to the manufacturers protocol. Fmoc-amino acids (Novabiochem) were dissolved in N-methylpyrrolidinone (NMP) containing 0.3M hydrobenzotriazole (HOBt) to a final concentration of 0.3M. Coupling was done by activating with diisopropylcarbodiimide (DIC). De-protection of the Fmoc group was done by 20% piperidine in NMP. The individual sequences were designed using the array synthesiser software. After synthesis, the protecting groups were removed by treatment of the sheets with 95% trifluoroacetic acid (TFA) containing triisopropylsilane (TIPS) for 60 min. The sheets were washed with dichloromethane (DCM) and N-methylpyrrolidinone (NMP) and finally with water.

### Labelling of Antibody

The stock of the 4F3B mAb was subjected to gel-filteration chromatography, using a NAPS column (GE Healthcare) to replace the buffer with 1% NaHCO₃ according to the manufacturer recommendations. This was followed by adding 25 mole equivalents of 5(6)-carboxyfluorescein N-hydroxysuccinimide ester (Sigma, #C1609) dissolved in DMSO. The coupling reaction continued for 2 hours followed by gel-filtration against the washing buffer (50 mM TRIS, pH7.4, 0.15M NaCl, 0.1M ArgHCl, 0.05% Tween 20) in order to removed uncoupled fluorescein.

### Screening of Arrays

Screening was performed with 10 μl of fluorescein-labelled 4F3B in 30 ml incubation buffer (0.5% BSA, 50 mM TRIS, pH7.4, 0.15M NaCl, 0.1M ArgHCl, 0.05% Tween 20). The sheets were incubated for 1-2 hours, followed by washing five times with the washing buffer. The sheets were scanned using a laser scanner (Typhoon 9410, GE Healthcare) and the image file (.gel format) was analysed using an ArrayPro Analyzer (Media Cypernetics). The fluorescence intensity was measured and transformed into digits which were exported to GraphPad Prism 5 for further analysis.

### Results

The array analysis revealed that the mAb 4F3B binds to the regions corresponding to amino acid residues 29-48 and 129-146.

The structure of OSCAR has not yet been solved. Using an available 3D structure of KIR2DL2 as a template, a homology model of OSCAR-ECD was built. According to the model, the binding regions for mAb 4F3B are located in each of the Ig-like domains. The technique used here allows a preliminary prediction of linear epitopes. For accurate epitope mapping techniques such as hydrogen exchange and mass spectrometry (HX-MS) and/or X-ray crystallography of complexes between OSCAR and specific mAbs or fractions thereof (e.g. Fab fragments) can be employed.

An OSCAR variant having a Cys-to-Ala mutation in position 83 has been produced. This C83A OSCAR variant can potentially be useful for protein structure determination (e.g. X-ray analysis), protein-ligand interface determination and epitope mapping.

### Example 8

Inhibition of ColI-Induced DC Maturation and Cytokine Release by Anti-OSCAR mAbs Brand et al (Eur. J. Immunol., 1998, 28: 1673-1680) have shown that Collagen I induces maturation of monocyte-derived DC (moDC). These cells exhibited allostimulatory capacity. However, DC receptors mediating ColI-induced effects were not identified. Merck et al (Blood, 2005, 105: 3623-3632) have shown that cross-linking of OSCAR with specific antibodies can induce maturation of moDC. As ColI was identified as an interaction partner for OSCAR, we hypothesised that ColI-induced maturation of moDC is mediated by OSCAR. We have developed an assay allowing to test this hypothesis. It was found that immature moDC plated on un-treated (i.e. having neutral surface charges) virgin polysterene multi-well plates (e.g. from IWAKI) were best suited for coating with ColI providing maturation of moDC. Blocking of OSCAR with polyclonal antibodies abolished maturation of moDC plated on ColI. We therefore concluded that the assay was suitable as a functional screen for neutralizing anti-OSCAR mAbs.

### Methods

Human PBMC were isolated as described in Example 1. Monocytes were purified from PBMC using MACS CD14⁺ beads (Miltenyi MicroBeads, #130-050-201). The monocytes were seeded at a cell density of 2×10⁶ cells/ml and cultured for 3 days in the presence of 12.5 ng/ml IL-4 (R&D Systems, #204-IL-010) and 25 ng/ml GM-CSF (eBioscience, #14-8339) at 2×10⁶ cells/ml for 3 days. The monocytes differentiated into immature DC, which were non-adherent cells of irregular shape that expressed high levels of DC-SIGN. The immature DC were pre-incubated for 1 h with anti-OSCAR or control antibodies and subsequently seeded into plates (IWAKI, #1820-024) coated with ColI (Sigma, #C5483) or FCS (control) and incubated overnight. The maturation state of the DC (i.e. expression of HLA-DR, CD83, CD86 and DC-SIGN) was assessed by flow cytometry. Secretion of cytokines was analysed by ELISA (R&D systems). TNF-α and IL8 were identified as the cytokines providing the best window (ColI versus FCS treated DCs) and thus were chosen as readouts in the screen. All mAbs and ColI used in the assay were endotoxin-free. DC plated onto FCS-coated plates were used to control for agonistic properties of mAbs.

### Results

All anti-OSCAR mAbs which showed binding human monocytes were tested for inhibitory properties in this assay at multiple doses. IL8 and TNF-α concentrations were plotted as a function of log [conc. of mAb in nM]. The curves were fitted using the “log [inhibitor] vs. response-variable slope” fit (GraphPad Prism 5 software). IC₅₀ values were defined as the concentration of mAb inhibiting IL8 release to 50% of the level induced by ColI. Isotype control IgGs had no inhibitory activity. Several anti-OSCAR mAbs with comparable affinities did not inhibit cytokine release. All mAbs were screened using moDC from several donors. Table 6 summarises the IC₅₀ values which vary between donors.

TABLE 6
IC₅₀ values (IL-8) for different anti-OSCAR mAbs

| IC50 (nM) | Donor 1 | Donor 2 | Donor 3 | Donor 4 | Donor 5 | Donor 6 |
|---|---|---|---|---|---|---|
| Bin 1 | | | | | | |
| 4F3B | 0.19 | n.d. | 0.40 | 0.12 | 0.43 | 0.24 |
| 16F56A2 (Hu) | n.t. | n.t. | n.t. | 0.06 | 0.10 | 0.21 |
| 16F119A5 | n.t. | n.t. | n.t. | 0.02 | 0.46 | 1.12 |
| (Hu) | | | | | | |
| 13F5A5 (Hu) | 2.0 | n.d. | n.d. | <0.01 | 0.23 | 0.06 |
| 13F56A6 (Hu) | n.t. | n.t. | 1.4 | 1.54 | 2.0 | 1.88 |
| Bin 2 | | | | | | |
| 7F1A1 | <0.1 | <0.1 | 0.1 | 0.03 | n.t. | n.t. |
| 7F21A1 | n.d. | 0.02 | 0.64 | 0.11 | n.t. | n.t. |
| 3F7A9 | 0.16 | 0.06 | 0.41 | 0.06 | 0.05 | 0.10 |
| 11.1CN5 | n.t. | n.t. | n.t. | 0.31 | >20 | 0.84 |
| Bin 3 | | | | | | |
| 7F14A4 | 0.23 | n.d. | 0.62 | n.t. | n.t. | n.t. |
| 7F10A1 | 0.49 | n.d. | 1.59 | 2.06 | n.t. | n.t. |
| Bin 4 | | | | | | |
| 7F6A1 | n.d. | n.d. | 0.91 | n.t. | 2.49 | 1.28 |
| 6F6B | n.t. | n.t. | n.t. | 0.86 | >20 | 6.56 |
| 6F3A | n.t. | n.t. | n.t. | 0.69 | >20 | 8.06 |

n.d. not determined.
n.t. not tested.

### Conclusions

Nine mouse- and four human inhibitory mAbs were identified representing 33% and 36%, respectively, of mAbs which demonstrated strong binding to monocytes. The inhibitory anti-OSCAR mAbs had no agonistic properties. The comparative potency of the mAbs is the same even though the IC₅₀ values vary between donors.

### Example 9

Anti-OSCAR mAb Inhibit DC Maturation and Hence their Ability to Promote T-Cell Proliferation in an Allogenic Mixed Lymphocyte Reaction.

The ability of the colI-matured DC to promote T-cell proliferation has been assessed using an allogenic mixed lymphocyte reaction (MLR).

### Method of Allogenic Mixed Lymphocyte Reaction (MLR)

### (1) Purification of CD14+ Cells, Differentiation and Maturation of DC

CD14+ monocytes were purified from buffy coats of healthy donors. The cells were enriched using RosetteSep Human Monocyte Enrichment Cocktail (StemCell Technology, #15068) according to the manufacturers protocol. Residual erythrocytes were lysed using RBC lysis buffer (eBioscience, #00-4333-57) for 10 min at room temperature. Monocytes were washed twice with the buffer containing PBS, 2% FBS and 10 mM EDTA, collected by centrifugation and re-suspended at the concentration of 6.7×10⁵ cells/ml in the DC differentiation medium containing RPMI 1640 with 2 mM L-glutamine (Glutamax™, Gibco, #61870), 10% FBS, 50 ng/ml human recombinant GM-CSF (R&D, #215-GM) and 50 ng/ml human recombinant IL-4 (PeproTech, #200-04). 1.2×10⁷ cells were seeded onto a 92×17 mm tissue culture plate (Nunc, #150350) and cultured for five days in 5% CO₂ at 37° C. The medium was renewed after two days of culturing.

After five days, non-adherent immature DC (iDC) were collected, extensively washed in a growth medium without GM-CSF and IL-4 (RPMI 1640 with 2 mM L-glutamine, 10% FBS), and re-suspended at 4×10⁵/ml in the same growth medium. An aliquot of cells was taken for analysis of the surface markers expression using flow cytometry (see FACS analysis). The cells were pre-incubated for 30 min with anti-OSCAR mAbs prior to transfer to 24 well microplates (IWAKI, #1820-024), which were coated for 3 hours at room temperature with 300 μl/well of 10 μg/ml human ColI (Sigma, #C5483) and rinsed thoroughly with PBS, or to the uncoated wells. Unless indicated, 4×10⁵ cells per well were used. The DC growth/survival factors (50 ng/ml GM-CSF and 50 ng/ml IL-4) or inducers of maturation (5 μg/ml LPS, Sigma, #SC-3535) were added to the control wells. The cells were cultured for two days in 5% CO₂ humidified cell incubator at 37° C. Non-adherent cells were collected, extensively washed and resuspended in RPMI 1640 supplemented with 10% FBS and 2 mM L-glutamine (further referred as “T-cell growth medium”) for further use in MLR. Expression of cell surface markers was analysed by flow cytometry (see FACS analysis). Detection of cytokines in the conditioned medium was performed using Bio-Plex multiplex analysis (see Analysis of Cytokine secretion).

### (2) Mixed Lymphocyte Reaction (MLR)

CD4⁺ human T-cells were isolated from buffy coats of healthy donors using RosetteSep Human CD4⁺ T cell Enrichment Cocktail (StemCell Technologies, #15062) according to the manufacturers protocol followed by Histopaque density gradient purification (Sigma, #10771). The CD4⁺ cells were recovered from the interphase, thoroughly washed with PBS/2% FCS and re-suspended in T-cell growth medium. The purity of the T-cells was analysed by flow cytometry (see FACS analysis).

To set up MLR, DC were mixed with allogenic CD4⁺ T-cells in a ratio 1:100 (10³ DC:10⁵ T-cells per well of a 96-well plate). The cells were cultured in 5% CO₂ humidified cell incubator at 37° C. for 5 days. T-cells cultured without DC served as negative control. At day 4 conditioned medium was collected for analysis of cytokine secretion (see Analysis of cytokine secretion). The cells were then pulse-labelled with 0.5 μCi/well ³H-Thymidine (Perkin Elmer, 14.4 Ci/mmol) for 18 hours. The cells were collected using a Tomtec cell harvester and lysed in distilled water. Incorporated thymidine was captured on glass fibre filters. The filters were air-dried for 20 hours at room temperature prior to quantification using a TopCounter scintillation counter (Perkin Elmer Topcount NXT).

### (3) FACS Analysis

The following antibodies were used for phenotype analysis of moDC:

Cocktail #1: CD14-APC (BD, #555399)/HLA-DR-FITC (BD, #335831)/Live/Dead Cell Staining DCS-Near-IR (Molecular Probes, # L10119)/OSCAR-PE (Beckton Coulter, clone 11.1CN5).

### Cocktail #2: CD86-PE/CD83-APC/209-PerCP-Cy5.5 (BD, #84366)

To estimate the purity of the isolated CD4⁺ T-cell population the following antibodies were used:

### Cocktail #1: CD4-FITC (BD, #555348)/CD8-PE (BD, #555635)/CD19-PE-Cy7 (BD, #557835)/CD3-APC (BD, #555335)/CD14-APC-H7 (BD, #641394).

Cocktail #2: CD4-APC (BD, #555349)/Live/Dead Cell Staining DCS-Near-IR (Molecular Probes cat# L10119).

The cells were analysed using an LSRII flow cytometer. The data were treated using FACS Diva and FlowJo softwares.

### (4) Analysis of Cytokine Secretion

The cytokine secretion was assessed using a Bio-Plex system for multiple cytokine analysis (BioRad). 50 μl of cell culture supernatants were analysed. Secreton of IL23(p19/p40) was analysed by ELISA (eBiosciences, #88-7239) with the capture anti-IL12p40 and detection anti-p19 Ab according to the manufacturers protocol. Data analysis was performed using the GraphPad Prism 5.0 software.

### Results

1. Col1 induced the maturation of DCs and cytokine release, which can be inhibited by anti-OSCAR mAbs (Table 7).

Non-adherent immature DC (iDC) differentiated from monocytes in the presence of GM-CSF/IL4 were transferred either onto the plates coated with ColI and maintained in the full growth medium, or onto uncoated plates in the presence of LPS (positive control, “classical” mature DC) or uncoated plates in the presence of GM-CSF/IL4 (negative control, to maintain the iDC phenotype). The expression of the maturation cell surface markers was analysed by FACS. Cells propagated on ColI were phenotypically similar to the classical LPS-matured DC (CD80^{high}/CD83^{high}/CD86^{high}/DC-SIGN⁺/CD14⁻), while the negative control cells remained CD80^{low}/CD83^{low}/CD86^{low}/DC-SIGN⁺/CD14⁻. The cytokine secretion profile of the ColI-matured DC was somewhat different from that of the LPS-matured DC. In contrast to the LPS-matured DC, the ColI-matured DC expressed IL23, but not IL12 and higher amounts of IL2 as compared to the LPS-matured DC. Multiple pro-inflammatory cytokines and chemokines were detected in supernatants from both, LPS- and ColI-matured DC. The ColI-induced cytokine release was inhibited by anti-OSCAR mAbs 4F3B and 3F7A9. The non-inhibitory anti-OSCAR mAb 9F11A2 served as an isotype control (Table 7).

2. ColI-matured DC stimulated T-cell proliferation and cytokine secretion in an allogenic MLR assay, which can be inhibited by anti-OSCAR mAbs (Table 8 and 9).

To address functionality of the ColI-matured DC, an allogenic mixed lymphocyte reaction has been set. ColI-matured DC stimulated T-cell proliferation at least as efficient as the classical LPS-matured DC. The proliferation of T-cells was significantly lower when the cells were co-cultured with DC treated with the anti-OSCAR mAbs 4F3B and 3F7A9 (Table 8). The cytokine secretion profile of the T-cells is shown in Table 9. Even though the DC used in MLR were not PFA-fixed, the T-cell growth medium does not support survival of DC, thus the contribution of the DC-derived cytokines is minimal (if any).

### Conclusion

These data suggest that anti-OSCAR mAbs inhibit DC maturation, and hence suppress their ability to promote an allogenic T-cell proliferation.

TABLE 7
Secretion of cytokines by DC

| | | | | ColI-DC | ColI-DC |
|---|---|---|---|---|---|
| | | | | 0.5 μg/ml | 5 μg/ml |
| | iDC | LPS-DC | ColI-DC | 4F3B | 4F3B |
| IL-2 | 33 ± 8 | 26 ± 5 | 28 ± 1 | 17 ± 1 | 28 ± 1 |
| IL-3 | 315 ± 22 | 571 ± 26 | 380 ± 4 | 569 ± 15 | 786 |
| IL-4 | 2 | 2 | 2 | 2 | 3 |
| IL-5 | 19 ± 17 | 170 ± 13 | 22 ± 3 | 3 | 2 |
| IL-6 | 2428 ± 2 | 22547 ± 364 | 8565 ± 176 | 1729 ± 98 | 543 ± 92 |
| IL-8 | 11406 ± 486 | 20286 ± 508 | 20167 ± 523 | 11809 ± 458 | 4279 ± 114 |
| IL-10 | 239 ± 48 | 12049 ± 126 | 2801 ± 186 | 86 ± 11 | 79 ± 2 |
| IL-12p40 | 536 ± 35 | 39407 ± 17771 | 46361 ± 406 | 596 ± 49 | 531 ± 61 |
| IL-12p70 | 72 ± 34 | 14287 ± 316 | 100 ± 6 | 40 ± 2 | 43 ± 6 |
| IL-23* | ND | 696 ± 9 | 272 ± 6 | ND | ND |
| IL-13 | 21 ± 11 | 1658 ± 7 | 1034 ± 34 | 11 | 9 |
| IL17 | 50 ± 6 | 51 ± 1 | 46 ± 8 | 50 | 62 ± 5 |
| INF | 78 ± 37 | 4937 ± 870 | 69 | 69 | 87 |
| MCP-1 | 1968 ± 121 | 1790 ± 83 | 2606 ± 51 | 207 ± 10 | 89 ± 12 |
| RANTES | 64 ± 15 | 4479 ± 193 | 2201 ± 132 | 55 | 34 ± 3 |
| TNF | 367 ± 101 | 44763 ± 1881 | 31108 ± 1967 | 78 | 61 |

| | ColI-DC | ColI-DC | ColI-DC | ColI-DC |
|---|---|---|---|---|
| | 0.5 μg/ml | 5 μg/ml | 0.5 μg/ml | 5 μg/ml |
| | 3F7A9 | 3F7A9 | 9F11A2 | 9F11A2 |
| IL-2 | 22 ± 1 | 19 | 26 ± 4 | 33 ± 3 |
| IL-3 | 517 | 525 ± 55 | 429 ± 7 | 460 ± 80 |
| IL-4 | 2 | 2 | 2 | 2 |
| IL-5 | 2 | 3 ± 1 | 22 | 21 ± 3 |
| IL-6 | 3176 ± 282 | 3658 ± 255 | 6750 ± 501 | 6589 ± 316 |
| IL-8 | 16274 ± 163 | 15378 ± 193 | 19911 ± 700 | 19751 ± 587 |
| IL-10 | 208 ± 26 | 176 ± 12 | 2330 ± 48 | 2231 ± 132 |
| IL-12p40 | 13398 ± 1279 | 3399 ± 233 | 46937 ± 2298 | 44221 ± 2396 |
| IL-12p70 | 50 ± 3 | 43 | 87 | 95 |
| IL-23* | 10 ± 5 | ND | 289 ± 40 | 228 ± 17 |
| IL-13 | 44 ± 6 | 19 ± 2 | 1039 ± 35 | 956 ± 68 |
| IL17 | 53 ± 2 | 56 ± 6 | 44 ± 5 | 52 ± 4 |
| INF | 78 ± 37 | 95 ± 12 | 95 ± 12 | 78 ± 12 |
| MCP-1 | 525 ± 37 | 453 ± 19 | 2561 ± 88 | 2405 ± 212 |
| RANTES | 62 ± 2 | 46 ± 1 | 2131 ± 53 | 1872 ± 32 |
| TNF | 217 ± 12 | 104 ± 12 | 28719 ± 2669 | 26543 ± 1310 |

Data represent mean ± SD (n = 3), pg/ml;
*IL-23 was measured using p40/p19 specific ELISA;
ND: not detectable

TABLE 8
Allogenic mixed lymphocyte reaction: proliferation
of T-cells stimulated by DC which were matured in
the presence of the indicated agents

| | CPM, mean ± SD (n = 3) |
|---|---|
| Immature DC | 27032 ± 5148 |
| LPS matured DC | 31155 ± 2768 |
| ColI matured DC | 38532 ± 2445 |
| ColI matured DC + 0.5 μg/ml 4F3B | 9498 ± 1546 |
| ColI matured DC + 5 μg/ml 4F3B | 7404 ± 1225 |
| ColI matured DC + 0.5 μg/ml 3F7A9 | 18437 ± 1451 |
| ColI matured DC + 5 μg/ml 3F7A9 | 15201 ± 3183 |
| ColI matured DC + 0.5 μg/ml 9F11A2 | 37780 ± 3396 |
| ColI matured DC + 5 μg/ml 9F11A2 | 40124 ± 3930 |
| No DC | 231 ± 31 |

TABLE 9
Secretion of cytokines by T-cells in allogenic mixed lymphocyte reaction

| | | | | | ColI-DC | ColI-DC |
|---|---|---|---|---|---|---|
| | Basal | iDC- | LPS-DC | ColI-DC | 0.5 μg/ml | 5 μg/ml |
| | level | induced | induced | induced | 4F3B | 4F3B |
| IL-2 | 23 ± 5 | 115 ± 30 | 229 ± 27 | 432 ± 101 | 118 ± 39 | 89 ± 11 |
| IL-3 | 675 ± 52 | 759 ± 27 | 701 ± 76 | 570 ± 305 | 725 ± 58 | 739 ± 73 |
| IL-4 | 2 | 4 ± 1 | 3 | 3 | 2 | 3 |
| IL-5 | 2 | 47 ± 14 | 419 ± 139 | 470 ± 146 | 70 ± 38 | 34 ± 16 |
| IL-6 | 28 ± 6 | 676 ± 38 | 1504 ± 256 | 687 ± 206 | 162 ± 58 | 196 ± 41 |
| IL-8 | 209 ± 82 | 15747 ± 451 | 3854 ± 69 | 7452 ± 1017 | 8907 ± 594 | 7801 ± 2189 |
| IL-10 | 22 ± 2 | 476 ± 83 | 469 ± 124 | 808 ± 551 | 56 ± 7 | 55 ± 19 |
| IL-12p40 | 290 ± 17 | 624 ± 104 | 964 ± 57 | 772 ± 260 | 383 ± 9 | 370 ± 18 |
| IL-12p70 | 34 ± 2 | 44 ± 15 | 47 ± 7 | 52 ± 6 | 34 ± 3 | 35 ± 3 |
| IL-13 | 4 ± 1 | 441 ±8 | 1310 ± 246 | 1755 ± 90 | 285 ± 136 | 163 ± 76 |
| IL17 | 44 ± 8 | 59 ± 4 | 129 ± 40 | 76 ± 23 | 55 ± 6 | 59 ± 3 |
| INFg | 41 ± 7 | 202 ± 75 | 718 ± 374 | 1113 ± 317 | 82 ± 5 | 71 ± 7 |
| MCP-1 | 8 ± 3 | 428 ± 53 | 23 ± 1 | 31 ± 5 | 550 ± 317 | 859 ± 224 |
| RANTES | 79 ± 39 | 531 ± 87 | 803 ± 52 | 1281 ± 523 | 167 ± 56 | 181 ± 62 |
| TNFa | 54 ± 4 | 561 ± 63 | 356 ± 104 | 495 ± 91 | 298 ± 24 | 281 ± 39 |

| | ColI-DC | ColI-DC | ColI-DC | ColI-DC |
|---|---|---|---|---|
| | 0.5 μg/ml | 5 μg/ml | 0.5 μg/ml | 5 μg/ml |
| | 3F7A9 | 3F7A9 | 9F11A2 | 9F11A2 |
| IL-2 | 165 ± 54 | 100 ± 33 | 393 ± 31 | 419 ± 153 |
| IL-3 | 593 ± 236 | 737 ± 62 | 698 ± 16 | 507 ± 188 |
| IL-4 | 3 | 2 | 3 | 3 |
| IL-5 | 205 ± 75 | 106 ± 15 | 385 ± 148 | 446 ± 53 |
| IL-6 | 140 ± 69 | 126 ± 12 | 986 ± 255 | 806 ± 273 |
| IL-8 | 8508 ± 2079 | 6666 ± 749 | 8907 ± 594 | 7801 ± 2189 |
| IL-10 | 378 ± 96 | 132 ± 59 | 869 ± 171 | 990 ± 253 |
| IL-12p40 | 388 ± 151 | 410 ± 41 | 1025 ± 244 | 725 ± 194 |
| IL-12p70 | 40 ± 5 | 38 ± 3 | 98 ± 34 | 116 ± 74 |
| IL-13 | 650 ± 288 | 213 ± 21 | 1840 ± 310 | 1924 ± 179 |
| IL17 | 65 ± 4 | 63 ± 12 | 104 ± 13 | 92 ± 9 |
| INFg | 84 ± 40 | 82 ± 46 | 3667 ± 1331 | 1569 ± 882 |
| MCP-1 | 65 ± 24 | 148 ± 62 | 79 ± 10 | 44 ± 20 |
| RANTES | 340 ± 60 | 168 ± 56 | 1448 ± 397 | 1289 ± 47 |
| TNFa | 228 ± 98 | 228 ± 34 | 713 ± 68 | 675 ± 278 |

Data represent mean ± SD (N = 3), pg/ml

### Example 10

Anti-OSCAR mAbs Reduce Viability of DC Propagated on Col

ColI supported survival of iDC at the conditions of growth factor withdrawal. It has been investigated whether the effect is mediated by OSCAR.

### Methods

iDC were differentiated from CD14⁺ monocytes as described in Example 9. After five days, non-adherent iDC were collected and extensively washed to remove traces of GM-CSF and IL4. The cells were re-suspended at 2.5×10⁶ cells/ml in the growth medium containing RPMI 1640 with Glutamax™, (Gibco, #61870) supplemented with 10% FBS. Prior to seeding, aliquots of 5×10⁵/200 μl cells were pre-incubated for 30 min at 37° C. with 300 μl of anti-OSCAR mAbs (diluted with growth medium) or with 300 μl of growth medium without mAb as untreated control. After incubation the cells were transferred to 24 well microplates (IWAKI, #1820-024), which were coated for 3 hours at room temperature with either 300 μl/well of 10 μg/ml human ColI (Sigma, #C5483) or 20 μg/ml fibronectin (Sigma, #F0895) and rinsed thoroughly with PBS, or to the uncoated wells. 500 μl of the growth medium was added per well. As positive control, 50 ng/ml GM-CSF was added to the indicated wells. The cells were propagated for three days in a 5% CO₂ humidified cell incubator at 37° C. Metabolic activity was analysed as a surrogate marker of cell viability using Alamar Blue dye (Invitrogen, # Da11025) according to the manufacturers protocol. Fluorescence was measured at excitation 544 nm/emission 590 nm using a NovoStar fluorescence plate reader (BMG). Data analysis was performed using the GraphPad Prism 5.0 software.

### Results

The effect of ColI-OSCAR interaction on the viability of DC under conditions of growth factor withdrawal has been addressed. Metabolic activity of the cells was used a surrogate marker of cell viability. Monocyte-derived iDC cultured on ColI were at least as viable as the cells cultured in the presence of the growth factor GM-CSF. In contrast, the cells plated onto another matrix protein, fibronectin, or onto un-coated plastic demonstrated poor viability. Anti-OSCAR mAbs suppressed metabolic activity of the DC plated on ColI indicating that the survival-promoting effect of ColI is mediated by OSCAR (Table 10).

### Conclusion

ColI-OSCAR interaction promotes the survival of DCs under the conditions of grow factor withdrawal and the effect can be suppressed by anti-OSCAR mAbs.

TABLE 10
ColI-OSCAR interaction supports survival of DC
under conditions of growth factor withdrawal

| | RFU (Alamar Blue, 544/590 nm) |
|---|---|
| | Mean ± SD (n = 3) |
| Uncoated | 1763 ± 111 |
| Uncoated; +GM-CSF | 2629 ± 112 |
| Fibronectin | 1644 ± 529 |
| ColI | 3407 ± 173 |
| ColI + 0.1 μg/ml 3F7A9 | 1115 ± 61 |
| ColI + 1 μg/ml 3F7A9 | 1084 ± 76 |
| ColI + 0.1 μg/ml 4F3B | 1344 ± 78 |
| ColI + 1 μg/ml 4F3B | 1061 ± 60 |
| ColI + 0.1 μg/ml IgG₁ | 3072 ± 389 |
| ColI + 1 μg/ml IgG₁ | 3046 ± 219 |

### Example 11

Anti-OSCAR mAbs Inhibit ColI/Col II-Induced Cytokine Release from RA Synovial Fluid cells

It has been shown that in vitro ColI stimulate secretion of pro-inflammatory cytokines by SFMC of RA patients (Jeng K C et a.l (1995) Immunol. Lett. 45 (1-2): 13-17). Since in RA joints ColI and ColII can be exposed due to progressive tissue damage, contact with immune cells and their activation by these matrix proteins may have physiological implications. Here, we have tested whether activation of RA synovial fluid cells by ColI/ColII is, at least in part, mediated by OSCAR and can be inhibited by anti-OSCAR mAbs.

### Preparation of RA Synovial Fluid Cells and Isolation of RA SFMCs

Synovial fluid from knee joints of RA patients was collected by needle aspiration at Peking University People's Hospital Hospital (Beijing, China). The RA synovial fluid cells were collected by centrifugation, re-suspended in RPMI 1640 medium and purified by Ficoll density gradient centrifugation. RA synovial fluid mononuclear cells (RA SFMCs) were recovered from the interphase, washed twice with PBS and re-suspended in RPMI 1640 supplemented with 10% FBS and 2 mM L-glutamine.

### Analysis of ColI-Induced Cytokine Secretion

OSCAR ligand ColI (Millipore, #cc050), ColII (Millipore, #cc052), or the control protein collagen V (Millipore, #cc077), were immobilized on a 96-well plate (Costar, #3361) at 100 μl/well of 10 μg/ml protein solution in PBS for 4 h at 37° C. RA synovial fluid cells (3×10⁵1200 μl per well) or RA SFMCs (1×10⁵/200 μl per well) were pre-incubated with the anti-OSCAR mAbs or isotype control mAbs for 1 h prior to be transferred onto the collagen-coated plate. After 24 hours incubation, the cell culture supernatants were analysed by ELISA for the presence of TNF-α, IL-6 and IL-8.

### Results

Plate-bound ColI/ColII induced robust release of TNFα, IL-6 and IL-8 from RA synovial fluid cells as well as from the isolated RA SFMCs, which can be inhibited by anti-OSCAR mAbs. For example, two mouse anti-OSCAR mAbs 3F7A9-1 and 4F3B, but not the isotype control anti-OSCAR mAb 9F11A2 (mlgG1), inhibited ColI-induced TNFα and IL-8 secretion from RA SFMCs. The inhibition was not dose-dependent within the tested range of mAb concentrations presumably due to a high potency of these mAbs: the maximal effect was reached already at 0.1 μg/ml. (Table 11 and 12).

Furthermore, by using RA synovial fluid cell, 4F3B (murine mAb) and 13F5A5 (Human mAb), but not their corresponding isotype control, inhibited ColI/ColII-induced inflammatory cytokine release (Table 13). These data suggest that blocking of the interaction between OSCAR and ColI/ColII can prevent activation of the myeloid cells present in the RA synovial fluid.

### Conclusion

Anti-OSCAR mAbs inhibit ColI/ColII-induced inflammatory cytokine release from RA synovial fluid cells, suggesting blocking OSCAR can inhibit the induction of inflammatory responses in myeloid cells present in RA synovial fluids.

TABLE 11
Anti-OSCAR mAbs inhibit ColI-induced
IL-8 secretion from RA SFMCs

| anti-OSCAR | % of inhibition | | |
|---|---|---|---|
| mAbs | 0.1 μg/ml | 1 μg/ml | 10 μg/ml |
| 3F7A9-1 | 60 | 60 | 63 |
| 4F3B | 64 | 81 | 88 |
| 9F11A2 | 24 | 10 | 4 |

RA SFMCs: RA synovial fluid mononuclear cells

TABLE 12
Anti-OSCAR mAbs inhibit ColI-induced TNFα
secretion from RA SFMCs

| anti-OSCAR | % of inhibition | | |
|---|---|---|---|
| mAbs | 0.1 μg/ml | 1 μg/ml | 10 μg/ml |
| 3F7A9-1 | 91 | 93 | 91 |
| 4F3B | 95 | 100 | 100 |
| 9F11A2 | 25 | 13 | 0 |

RA SFMCs: RA synovial fluid mononuclear cells

TABLE 13
Anti-OSCAR mAbs inhibit ColI/ColII-induced TNFα
secretion from RA synovial fluid cells

| | |
|---|---|
| anti-OSCAR | % of inhibition |

| mAbs (10 μg/ml) | ColI-induced TNFα | ColII-induced TNFα |
|---|---|---|
| mIgG1 | 26 | 15 |
| 4F3B | 90 | 95 |
| 9F11A2-hIG1.1 | 20 | 20 |
| 13F5A5-hIgG1.1 | 100 | 85 |

### Example 12

### Osteoclast Formation Assay

Osteoclast formation assays can be used to detect if an OSCAR mAb has the ability to reduce bone erosion. The mAbs with the ability to inhibit bone erosion by at least 10%, preferably at least 20%, preferably at least 25%, preferably at least 30%, preferably at least 40%, preferably at least 50%, preferably at least 75%, and most preferably at least 90%. Osteoclasts are multinucleated cells derived from myeloid progenitors that are capable of eroding bone. A standard protocol for generating osteoclasts in vitro is to culture monocytes for 7-14 days in the presence of exogenous recombinant M-CSF and soluble RANKL, both factors are required and sufficient for OC formation. During such culture conditions monocytes typically change characteristics as follows:
Day 0-6: low levels of cell fusion and TRAP activity
Day 6-8: preosteoclast characteristics with evident cell fusion, increased TRAP activity, and cathepsin K expression
Day 9-: mature osteoclast characteristics, multinucleation, bone resorption, high TRAP activity, and high cathepsin K expression.
In this example, we tested the effect of anti-OSCAR human mAbs in such a standard osteoclast formation assay using PB CD14+ monocytes from peripheral blood of healthy donors.

### Methods

### (1) Purification of Monocytes

Primary human monocytes are purified by a positive selection method using anti-CD14 mAb coated microbeads. Briefly, PBMCs are isolated from buffy coats using Ficoll density gradient centrifugation. Thereafter, monocytes are isolated using CD14 microbeads followed by MACS column separation (Miltenyi Biotec, #130-050-201) according to the manufacturers recommendations.
The OC formation assay is set up in 96 well plate formats.

### (2) OC Formation Assay

The OC formation assay is set up in a 96 well plate format. A starting concentration of 60 000 monocytes/well is used. The cells are cultured either on uncoated plastic in a final volume of 200 μl of the growth medium alone (IMDM, 2% human serum, 10% FCS, P/S, 2 mM L-glutamine), or in the growth medium supplemented with recombinant human M-CSF (25 ng/ml) and soluble RANKL (100 ng/ml).
To evaluate whether anti-OSCAR mAbs impact osteoclast formation and/or active bone resorption, each mAb is added at 10 μg/ml at the initiation of culture. The mAbs with hlgG1.1 backbone used in this assay are non-depleting antibodies.
At the end of the assay (day 9-12), supernatants are harvested and the adherent cells grown on plastic and bone slices are washed 3 times with 2 ml PBS and fixed with 4% paraformaldehyde (PFA) in PBS for 5 min. After washing in ddH₂O the cells are stained for TRAP using a leukocyte acid phosphatase kit and counter-stained with hematoxyline according to instructions provided by the manufacturer (Sigma-Aldrich).

### Results

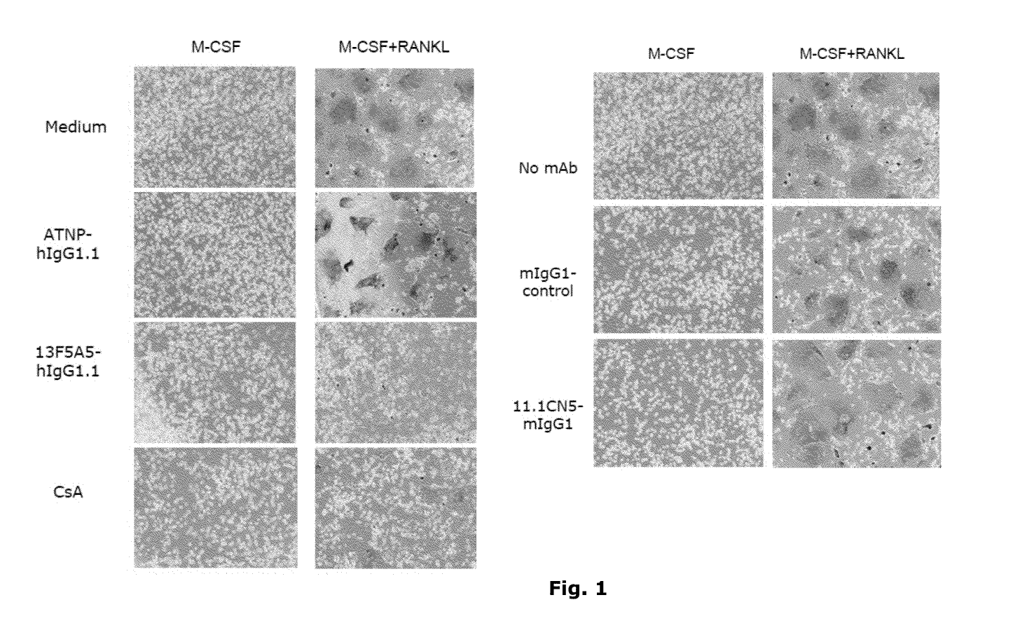

After 9 days culture, normal CD14⁺ monocytes were able to differentiate into robust mature osteoclasts in the presence of both M-CSF and RANKL. Cells treated with 13F5A5-hlgG1.1 and Cycrosporine A (CsA) showed inhibitory effect on osteoclastogenesis, whereas 11.1CN5 (Beckman Coulter, #PN A24987), 9F11A-hlgG1.1 (non-blocking mAb) and isotype control mAb ATNP-hlgG1.1 showed no impact on osteoclast formation. Osteoclast formation/activity was measured by TRAP (Osteoclast marker) staining (FIG. 1) and soluble TRAP5B secretion (Table 14).

### Conclusion

Anti-OSCAR mAb (i.e. 13F5A5-hlgG1.1) inhibited Osteoclast formation from normal monocytes, suggesting blocking of OSCAR will reduce osteoclastogenesis and bone erosion in RA.

TABLE 14
Anti-OSCAR mAb 13F5A5 inhibited TRAP5b production
from osteoclasts formed from normal monocytes

| Tested mAbs | % inhibition on TRAP5b |
|---|---|
| ATNP-hIgG1.1 | 5 |
| 13F5A5-hIgG1.1 | 40 |
| mIgG1 | 10 |
| 11.1CN5 | 0 |
| CsA | 45 |

### Example 13

### Competitive Binding-SPR Analysis of Competition Between Anti-OSCAR mAb and ColI for Binding to OSCAR

The experiment was performed using a Biacore T 100 instrument (GE Healthcare). Human collagen 1 (Sigma, # C5483) and human collagen 2 (Millipore #CC052) were diluted with 10 mM NaOAc, pH 4.5 and immobilized on a Biacore CM3 sensor chip (GE Healthcare) using amine coupling chemistry (GE Healthcare). Immobilization was performed according to the standard procedure, but at 5° C. in order to prevent aggregation collagens, with immobilized levels of ca 250 RU on flow cells (FC) 2 and 4. A blank immobilization was performed on FC 1 & 3, which were used as reference cells. HBS-EP buffer (10 mM HEPES, 150 mM NaCl, 3 mM EDTA, 0.05% Polysorbat P20, pH7.4) was used as running buffer for the immobilization. Buffer containing HBS-EP/+0.1% BSA was used for the competition assay. The competition experiment was performed at 5° C. at a flow rate of 50 μl/min. OSCAR-ECD (SEQ ID NO 2) was used as analytes at a concentration of 50 nM. OSCAR was mixed with various concentrations of anti-OSCAR antibodies ranging from 0-10 μg/ml prior to injection. Binding curves were measured for 360 s followed by 240 s of dissociation. The chip surface was regenerated for 2×20 s with 10 mM glycine, pH 2.0 between assays.

### Results

Human collagen I and II were immobilized to the surface of a Biacore sensor chip by amine coupling. Binding of OSCAR-ECD (SEQ ID NO 2) to collagens 1 & 2 was studied in the presence or absence of anti-OSCAR antibodies. It was hypothesized that an antibody inhibiting binding of OSCAR to collagen would lead to a concentration-dependent loss of signal, whereas a non-inhibiting antibody would enhance the signal due to the increased mass of the OSCAR-antibody complex. Of the 15 tested antibodies, 13 fell into one of the two groups. As shown in Table 15, mAbs 16F119, 16F56, 13F56A6, 13F5A5, 16F18, 4F3B and 6F6B inhibited binding of OSCAR to collagen with very little binding left in the presence of 10 μg/ml antibody. In contrast, 7F10A1, 7F14A1, 3F7A9, 7F21A1, 7F1A1 and the prior art antibody 11.1CN5 did not interfere with binding since a concentration-dependent increase of the signal was detected.

### Conclusions

Anti-OSCAR mAbs in epitope Bin #1 and #4, but not Bin #2 (incl. 11.1CN5) or #3 compete the binding of Collagens to OSCAR. The ligand competing data functionally differentiate the mAbs between the four epitope Bins.

TABLE 15
The effect of anti-OSCAR mAbs from the four different
epitope binds on the binding of OSCAR-ECD to
ColI/ColII by SPR analysis.

| | | collagen I | | Collagen II | |
|---|---|---|---|---|---|
| | | Binding | binding | Binding | binding |
| Epitope | | w/o | with 10 μg/mL | w/o | with 10 μg/mL |
| bin# | mAb | mAb | mAb | mAb | mAb |
| 1 | 4F3B | 10.9 | 0.7 | 17.8 | 6.7 |
| 1 | 13F5A5 | 9.4 | −1.4 | 14.3 | 1.3 |
| 1 | 13F56A6 | 9.3 | −2.1 | 13.7 | −1.4 |
| 1 | 16F18 | 8.8 | −0.3 | 12.2 | 0.1 |
| 1 | 16F56 | 8.4 | −3.2 | 10.9 | −1.9 |
| 1 | 16F119 | 8.4 | −3.4 | 10.6 | −1.5 |
| 2 | 3F7A9 | 10.1 | 132.2 | 17.6 | 246.4 |
| 2 | 7F1A1 | 10.8 | 144.6 | 17.6 | 335.4 |
| 2 | 7F21A1 | 11 | 143.5 | 18.2 | 319.1 |
| 2 | 11.1CN5 | 10.9 | 145.9 | 18.3 | 304 |
| 3 | 7F10A1 | 10.5 | 55 | 17.6 | 102.4 |
| 3 | 7F14A1 | 10.7 | 56.3 | 18.1 | 83.6 |
| 4 | 6F3A | 11.3 | 9.5 | 18.4 | 13.8 |
| 4 | 6F6B | 11.4 | 1.7 | 18 | 1 |
| 4 | 7F6A1 | 11.1 | 19.2 | 17.2 | 19.3 |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. An isolated antagonistic OSCAR antibody comprising a heavy chain comprising a heavy chain CDR1, a heavy chain CDR2, and a heavy chain CDR3 and a light chain comprising a light chain CDR1, a light chain CDR2, and a light chain CDR3, the heavy chain CDR1 comprising an amino acid sequence SYWIV of SEQ ID NO:22; the heavy chain CDR2 comprising an amino acid sequence SIYPGDSDARYSPSFQG of SEQ ID NO:22; the heavy chain CDR3 comprising an amino acid sequence QKITMVRGVIITPLDGMDV of SEQ ID NO:22; the light chain CDR1 comprising an amino acid sequence RASQSVSSSYLA of SEQ ID NO:21; the light chain CDR2 comprising an amino acid sequence GASSRAT of SEQ ID NO:21; and the light chain CDR3 comprising an amino acid sequence QQYGSSPLT of SEQ ID NO:21.

2. The isolated antagonistic OSCAR antibody of claim 1, wherein the heavy chain comprises an amino acid sequence that is at least 90% identical to SEQ ID NO:22, and wherein the light chain comprises an amino acid sequence that is at least 90% identical to SEQ ID NO:21.

3. The isolated antagonistic OSCAR antibody of claim 2, wherein the heavy chain comprises an amino acid sequence of SEQ ID NO:22, and wherein the light chain comprises an amino acid sequence of SEQ ID NO:21.

4. A pharmaceutical composition comprising an antagonistic OSCAR antibody and a pharmaceutically acceptable excipient, the antagonistic OSCAR antibody comprising a heavy chain comprising a heavy chain CDR1, a heavy chain CDR2, and a heavy chain CDR3 and a light chain comprising a light chain CDR1, a light chain CDR2, and a light chain CDR3, the heavy chain CDR1 comprising an amino acid sequence SYWIV of SEQ ID NO:22; the heavy chain CDR2 comprising an amino acid sequence SIYPGDSDARYSPSFQG of SEQ ID NO:22; the heavy chain CDR3 comprising an amino acid sequence QKITMVRGVIITPLDGMDV of SEQ ID NO:22; the light chain CDR1 comprising an amino acid sequence RASQSVSSSYLA of SEQ ID NO:21; the light chain CDR2 comprising an amino acid sequence GASSRAT of SEQ ID NO:21; and the light chain CDR3 comprising an amino acid sequence QQYGSSPLT of SEQ ID NO:21.

5. The pharmaceutical composition of claim 4, wherein the heavy chain comprises an amino acid sequence of SEQ ID NO:22, and wherein the light chain comprises an amino acid sequence of SEQ ID NO:21.

6. A method of treating an inflammatory disease comprising administering to a patient in need thereof an antagonistic OSCAR antibody comprising a heavy chain comprising a heavy chain CDR1, a heavy chain CDR2, and a heavy chain CDR3 and a light chain comprising a light chain CDR1, a light chain CDR2, and a light chain CDR3, the heavy chain CDR1 comprising an amino acid sequence SYWIV of SEQ ID NO:22; the heavy chain CDR2 comprising an amino acid sequence SIYPGDSDARYSPSFQG of SEQ ID NO:22; the heavy chain CDR3 comprising an amino acid sequence QKITMVRGVIITPLDGMDV of SEQ ID NO:22; the light chain CDR1 comprising an amino acid sequence RASQSVSSSYLA of SEQ ID NO:21; the light chain CDR2 comprising an amino acid sequence GASSRAT of SEQ ID NO:21; and the light chain CDR3 comprising an amino acid sequence QQYGSSPLT of SEQ ID NO:21.

7. The method of treating an inflammatory disease of claim 6, wherein the inflammatory disease is rheumatoid arthritis (RA).

8. The method of treating an inflammatory disease of claim 6, wherein the inflammatory disease is psoriatic arthritis.

9. The method of treating an inflammatory disease of claim 6, wherein the heavy chain comprises an amino acid sequence of SEQ ID NO:22, and wherein the light chain comprises an amino acid sequence of SEQ ID NO:21.

10. The method of treating an inflammatory disease of claim 9, wherein the inflammatory disease is rheumatoid arthritis (RA).

11. The method of treating an inflammatory disease of claim 9, wherein the inflammatory disease is psoriatic arthritis.

12. A method of treating a bone resorption related disorder comprising administering to a patient in need thereof an antagonistic OSCAR antibody comprising a heavy chain comprising a heavy chain CDR1, a heavy chain CDR2, and a heavy chain CDR3 and a light chain comprising a light chain CDR1, a light chain CDR2, and a light chain CDR3, the heavy chain CDR1 comprising an amino acid sequence SYWIV of SEQ ID NO:22; the heavy chain CDR2 comprising an amino acid sequence SIYPGDSDARYSPSFQG of SEQ ID NO:22; the heavy chain CDR3 comprising an amino acid sequence QKITMVRGVIITPLDGMDV of SEQ ID NO:22; the light chain CDR1 comprising an amino acid sequence RASQSVSSSYLA of SEQ ID NO:21; the light chain CDR2 comprising an amino acid sequence GASSRAT of SEQ ID NO:21; and the light chain CDR3 comprising an amino acid sequence QQYGSSPLT of SEQ ID NO:21.

13. The method of treating a bone resorption related disorder of claim 12, wherein the bone resorption related disorder is osteoarthritis.

14. The method of treating a bone resorption related disorder of claim 12, wherein the bone resorption related disorder is osteoporosis.

15. The method of treating a bone resorption related disorder of claim 12, wherein the heavy chain comprises an amino acid sequence of SEQ ID NO:22, and wherein the light chain comprises an amino acid sequence of SEQ ID NO:21.

16. The method of treating a bone resorption related disorder of claim 15, wherein the bone resorption related disorder is osteoarthritis.

17. The method of treating a bone resorption related disorder of claim 15, wherein the bone resorption related disorder is osteoporosis.


SEQUENCE LISTING (30 sequences)

SEQ ID NO: 1 - protein, 263 aa, homo sapiens
MALVLILQLLTLWPLCHTDITPSVPPASYHPKPWLGAQPATVVTPGVNVTLRCRAPQPAWRFGLFKPGEIAPLLFRDVSSELAEFFLEEVTPAQGGSYRCCYRRPDWGPGVWSQPSDVLELLVTEELPRPSLVALPGPVVGPGANVSLRCAGRLRNMSFVLYREGVAAPLQYRHSAQPWADFTLLGARAPGTYSCYYHTPSAPYVLSQRSEVLVISWEDSGSSDYTRGNLVRLGLAGLVLISLGALVTFDWRSQNRAPAGIRP

SEQ ID NO: 2 - protein, 209 aa, HOMO SAPIENS
DITPSVPPASYHPKPWLGAQPATVVTPGVNVTLRCRAPQPAWRFGLFKPGEIAPLLFRDVSSELAEFFLEEVTPAQGGSYRCCYRRPDWGPGVWSQPSDVLELLVTEELPRPSLVALPGPVVGPGANVSLRCAGRLRNMSFVLYREGVAAPLQYRHSAQPWADFTLLGARAPGTYSCYYHTPSAPYVLSQRSEVLVISWEDSGSSDYTR

SEQ ID NO: 3 - protein, 264 aa, homo sapiens
DITPSVPPASYHPKPWLGAQPATVVTPGVNVTLRCRAPQPAWRFGLFKPGEIAPLLFRDVSSELAEFFLEEVTPAQGGSYRCCYRRPDWGPGVWSQPSDVLELLVTEELPRPSLVALPGPVVGPGANVSLRCAGRLRNMSFVLYREGVAAPLQYRHSAQPWADFTLLGARAPGTYSCYYHTPSAPYVLSQRSEVLVISWEGEGPEARPASSAPGMQAPGPPPSDPGAQAPSLSSFRPRGLVLQPLLPQTQDSWDPAPPPSDPGV

SEQ ID NO: 4 - protein, 268 aa, homo sapiens
DITPSVAIIVPPASYHPKPWLGAQPATVVTPGVNVTLRCRAPQPAWRFGLFKPGEIAPLLFRDVSSELAEFFLEEVTPAQGGSYRCCYRRPDWGPGVWSQPSDVLELLVTEELPRPSLVALPGPVVGPGANVSLRCAGRLRNMSFVLYREGVAAPLQYRHSAQPWADFTLLGARAPGTYSCYYHTPSAPYVLSQRSEVLVISWEGEGPEARPASSAPGMQAPGPPPSDPGAQAPSLSSFRPRGLVLQPLLPQTQDSWDPAPPPSDPGV

SEQ ID NO: 5 - protein, 232 aa, artificial
  7F1A1 Light Chain
MKLPVRLLVLLWLTDARCDIQMTQSPASLSVSVGETVTITCRASEKIYSNLAWYQQKQGKSPQLLVYAATNLADGVPSRFSGSGSGTQFSLKINSLQPEDFGSYYCQHFWGIPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 6 - protein, 241 aa, artificial
  7F1A1 Heavy Chain
MGWSWVFLFLLSVTAGVHSQVQLQQSGAELVRPGTSVKVSCKASGYAFTNYMIEWVKKRPGQGLEWIGVINPGSGGTNYNEKFENKATLTADKSSSTAYMQLSSLTSDDSAVYFCARSLDFDGAWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDC

SEQ ID NO: 7 - protein, 235 aa, artificial
  7F6A1 Light Chain
MDLQVQIISFLLISASVIMSRGENVLTQSPAIMSASPGEKVTMTCSVSSSISYMHWYRQKSFTSPQLWIYDTSKLASGVPGRFSGSGSGISYSLTISSMEAEDVATYYCFQGSEYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 8 - protein, 238 aa, artificial
  7F6A1 Heavy chain
MDFGLSLIFLVLTLKGVQCDVKLVESGGGLVKPGGSLKLSCAASGFTFSSYVMSWVRQTPEKRLEWVATISSGGSYTYYPDSVKGRFTISRDNAKNTLYLQMSSLKSEDTAMYYCTREGGNSPDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDC

SEQ ID NO: 9 - protein, 238 aa, artificial
  7F9A1 Light Chain
METDTLLLWVLLLWVPGSTGDIVLTQSPASLAVSLGQRATISCRASQSVSTSNSSYMHWCQQKPGQPPKLLIKYASNLESGVPARFSGSGSGTDFTLNIHPVEEEDTATYYCQHSWEIPFTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 10 - protein, 242 aa, artificial
  7F9A1 Heavy Chain
MEWSWVFIFLLSGTAGVLSEVQLQQFGAELVKPGASVKISCKASGYTFTDYNMDWVKQSHGKSLEWIGDINPNYDSTTYNQKFKGKATLTVDKSSSTAYMELRSLTSEDTAVYYCARAYGNYDWWYFDVWGAGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDC

SEQ ID NO: 11 - protein, 235 aa, artificial
  7F10A1 Light Chain
MDFQVQIFSFLLISASVIMSRGQILLTQSPAIMSASPGEKVTMTCSASSSVSYMHWYQQKPGSSPKPWIYDTSNLASGFPARFSGSGSGTSYSLIISSMEAEDAATYYCHQRSSYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - protein, 242 aa, artificial
  7F10A1 Heavy Chain
MGWSWVFLFILSGTAGVHSQVQLQQSGAELARPGASVKLSCKASGYTFTDNYINWVKQRTGQGLEWIGEIYPGSGNTYYNEKFKGKATLTADKSSSTAYMQLSSLTSEDSAVYFCARSANRYDRGDFDYWGQGTPLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDC

SEQ ID NO: 13 - protein, 235 aa, artificial
  7F14A1 Light Chain
MDFQVQIISFLLISASVIISRGQIVLTQSPAIMSASPGEKVTMTCSASSSVNYMHWYQQKSGTSPKRWIYDTSNLASGVPTRFSGSGSGTSYSLTISSMEAEDAATYYCQQWSSDPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 14 - protein, 241 aa, artificial
  7F14A1 Heavy Chain
RLWLVLGLLFCLVAFPSCVLSQVQLKESGPGLVAPSQSLSITCSVSGFSLTSYGVHWVRQPPGKGLEWLGVMWAGGSTNYNSALMSRLSISKDNSKSQVFLKMNSLQTDDSAMYYCALQKGYGNYFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDC

SEQ ID NO: 15 - protein, 234 aa, artificial
  7F21A1 Light Chain
MSVLTQVLGLLLLWLTDARCDIQMTQSPASLSVSVGETVTITCRASEKIYSNLAWYQQKQGKSPQLLVYAATNLADGVPSRFSGSGSGTQFSLKINNLQSEDFGSYYCQHFWGTPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 16 - protein, 241 aa, artificial
  7F21A1 Heavy Chain
MGWSWVFLFLLSVTAGVHSQVQLQQSGAELVRPGTSVKVSCRSSGYAFTNYLIEWIKQRPGQGLEWIGVITPGSGGTNYNENFKDKATLTADKSSNTAYMQLTSLTSDDSAVYFCARSFDYDGAWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDC

SEQ ID NO: 17 - protein, 240 aa, artificial
  4F3B Light Chain
MESQILVLMSLLFWVSGTCGDIVMTQSPSSLTVTAGEKVTMSCKSSQSLLNSGNQKNYLTWYQQKPGQPPKVLIYWASTRESGVPDRFTGSGSGTDFTLTISSVQAEDLAVYYCQNDYSYPLTFGTGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 18 - protein, 238 aa, artificial
  4F3B Heavy Chain
MGWSGIFIFLFSVTAGVHSQVQLQQSGAELAKPGASVKMSCKASGFTFTSYWMHWVKQRPGQGLEWIGYINPSTDYTEYNQKFKDKATLTADKSSSTAYMQLSSLTSEDSAVYYCARRSPFAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDC

SEQ ID NO: 19 - protein, 234 aa, artificial
  3F7A9-1 Light Chain
MRAPAQILGILLLWFPGIKCDIKMTQSPSSMYASLGERVTITCKASQDINSFLSWLQQKPGKSPKTLIYRANRLVDGVPSRFSGSGSGQDYSLTISSLEYEDMGIYYCLQYDDFPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 20 - protein, 238 aa, artificial
  3F7A9 Heavy Chain
MGWSGVFLFLLSGTTGVHSEIQLQQSGPELVKPGASVKVSCKASGYVFTSYNMYWVKQSHGKSLEWIGYIDPYNGGTRYNQKFKGKATLTVDKSSSTAYMHLNSLTSEDSAVYYCARLDYYGNFYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDC

SEQ ID NO: 21 - protein, 109 aa, artificial
  13F5A5 Variable Light Chain
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIKR

SEQ ID NO: 22 - protein, 128 aa, artificial
  13F5A5 Variable Heavy Chain
EVQLVQSGAEVKKPGESLKISCKGSGYSFTSYWIVWVRQMPGKGLEWMGSIYPGDSDARYSPSFQGQVTISADKSISTAYLQWSSLKASDTAMYYCARQKITMVRGVIITPLDGMDVWGQGTTVTVSS

SEQ ID NO: 23 - protein, 108 aa, artificial
  13F56 Variable Light Chain
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPTFGPGTKVDIKR

SEQ ID NO: 24 - protein, 118 aa, artificial
  13F56 Variable Heavy Chain
QVQLVQSGAEVKKPGSSVKVSCKASGGTFSSYAISWVRQAPGQGLEWMGKIIPILGITNYAQKFQGRVTITADKSTSTAYMELSSLRSEDTAVYYCANPLTFDAFDIWGQGTMVTVSS

SEQ ID NO: 25 - protein, 108 aa, artificial
  16F56 Variable Light Chain
DIQMTQSPSFLSASVGDRVTITCRASQSISSYLNWYQQKPGKAPKLLIYAASSLQSGVPSRFSGSGSGTDFTLTISSLQPEDFATYYCQCGYSTPLTFGGGTKVEIKR

SEQ ID NO: 26 - protein, 123 aa, artificial
  16F56 Variable Heavy Chain
QVQLVQSGAEVKKPGSSVKVSCKASGGTFSNYAISWVRQAPGQGLEWMGRIIPILGIAHNAQKFQDRVTITADKSTSTAYMELSSLRSEDTAVYYCASPSSGKGLPYWFFDLWGRGTLVTVSS

SEQ ID NO: 27 - protein, 108 aa, artificial [sequence omitted]

SEQ ID NO: 28 - protein, 121 aa, artificial
  16F56 Variable Heavy Chain
QVQLVQSGAEVKKPGSSVKVSCKVSGGTFSNYAISWVRQAPGQGLEWMGRIIPILGIAAYAQRFRDRVTITADKFTSTVYMELSSLRSEDTAVYYCASGQLGTPYWYFDLWGRGTLVTVSS

SEQ ID NO: 29 - protein, 108 aa, artificial
  6F3A Variable Light Chain
DIVLTQSPATLSVTPGDSVSLSCRASQSITNNLHWYQQKSHESPRLLIKYASQSISGIPSRFRGSGSGTDFTLSINSVETEDFGVYFCQQSNTWPRTFGGGTKLEIKR

SEQ ID NO: 30 - protein, 123 aa, artificial
  6F3A Variable Heavy Chain
EVQLQQSGAELVKPGASVKLSCTASGFNIKDTYVHWVKQRPDQGLEWIGRIDPAIGNTKYDPKFRGKATITADSSSNTAYLQLSSLTSEDTAVYYCARLSYGNYGLDYSMDYWGQGTSVTVSS